(19) United States
(12) Patent Application Publication
(10) Pub. No.: US 2025/0019441 A1
(43) Pub. Date: Jan. 16, 2025

(54) TREATMENT OF CANCER WITH ANTI-ILT2 ANTIBODIES

(71) Applicants: BIOND BIOLOGICS LTD., Misgav Industrial Park (IL); GENZYME CORPORATION, Cambridge, MA (US)

(72) Inventors: Dana Haves Ziv, Bikat Beit HaKerem (IL); Liat Tevel, Rosh Ha’Ayin (IL); Enas Halumi, Kfar Manda (IL); Nyra Goldstein, Eschar (IL); Tsuri Peretz, Yokneam Ilit (IL); Ilana Goldshtein, Kiryat Motzkin (IL); Tal Gabay, Atlit (IL); Anna Fridam-Dror, Kibbutz Dalia (IL); Itay Friedman, Givatayim (IL); Sharon Hashmueli, Tel Aviv (IL); Motti Hakim, Kibbutz Gazit (IL); Ilana Mandel, Manof (IL); Avidor Shulman, Rakefet (IL); Yair Sapir, Manof (IL); Tehila Ben-Moshe, Tel Aviv (IL); Giovanni Abbadessa, Winchester, MA (US); Meijing Wu, Bedford, MA (US); Nigel Patrick Somerville Crawford, Califon, NJ (US); Rui Wang, Belmont, MA (US); Maria Agarwal, Bethesda, MD (US); Cecilia Deantonio, Cambridge (GB); Raymond Perez, Pennington, NJ (US); Ji Lin, Newton, MA (US)

(73) Assignees: BIOND BIOLOGICS LTD., Misgav Industrial Park (IL); GENZYME CORPORATION, Cambridge, MA (US)

(21) Appl. No.: 18/770,474

(22) Filed: Jul. 11, 2024

Related U.S. Application Data
(60) Provisional application No. 63/513,037, filed on Jul. 11, 2023.
(60) Provisional application No. 63/550,356, filed on Feb. 6, 2024.
(60) Provisional application No. 63/643,156, filed on May 6, 2024.

(51) Int. Cl. C07K 16/28; A61K 9/00; A61K 31/519; A61K 31/555; A61K 39/00; A61P 35/00

(52) U.S. Cl. CPC C07K 16/2818; A61K 9/0019; A61K 31/519; A61K 31/555; A61P 35/00; C07K 16/2863; A61K 2039/507; A61K 2039/54; A61K 2039/545; C07K 2317/565

(57) ABSTRACT

The present invention relates to treatment of cancer using an anti-human ILT2 monoclonal antibody. The treatment may additionally comprise pembrolizumab, optionally with chemotherapy, or cetuximab.

### CROSS-REFERENCE TO RELATED APPLICATION

This application claims priority to U.S. Provisional Patent Application 63/513,037, filed Jul. 11, 2023; U.S. Provisional Patent Application 63/550,356, filed Feb. 6, 2024; and U.S. Provisional Patent Application 63/643, 156, filed May 6, 2024. The disclosures of those priority applications are incorporated by reference herein in their entirety.

### SEQUENCE LISTING

The instant application contains a Sequence Listing which has been submitted electronically in XML format, is part of the specification, and is hereby incorporated by reference herein in its entirety. The electronic copy of the Sequence Listing, created on Jul. 3, 2024, is named 022548.US092.xml and is 29,035 bytes in size.

### BACKGROUND OF THE INVENTION

The glycoprotein receptor immunoglobulin-like transcript-2 (ILT2) is a surface glycoprotein inhibitory receptor expressed on a wide range of immune cells, including monocytes, dendritic cells, natural killer (NK), B cells, and T cells. ILT2 has been shown to bind to classical and nonclassical major histocompatibility complex (MHC) class I molecules, with a higher affinity to the nonclassical MHC-I molecule human leukocyte antigen-G (HLA-G). HLA-G is commonly expressed by solid tumors and plays an important role in immunotolerance by inhibiting cytolytic functions of NK cells and cytotoxic T-lymphocytes, and T-cell allo-proliferative responses. While HLA-G is believed to have an advantageous role in pregnancy, organ transplantation, and combatting autoimmune disease, its immunosuppressive effects may aid cancer growth by allowing cancer cells to evade the innate anti-cancer response.

The interaction between ILT2 and MHC-I (e.g., HLA-G) in the context of an immune reaction can lead to impairment of immune cell proliferation, differentiation, phagocytosis, cytotoxicity, cytokine secretion and chemotaxis, and to induction of regulatory cells and myeloid-derived suppressor cells (MDSC) or M2 type macrophages. These immunosuppressive effects identify the ILT2 signaling axis as a potential target for anti-cancer immunotherapies.

Some of the current cancer immunotherapies combat tumor progression by targeting proteins that aid in tumor immune evasion. However, such therapies are often inadequate due to primary tumor refractoriness and acquired tumor resistance. Thus, there remains a need for new and improved cancer immunotherapies.

### SUMMARY OF THE INVENTION

The present disclosure provides methods for cancer therapy using anti-human ILT2 antibody SAR444881 (i.e., BND-22) or a related antibody.

In some embodiments, the present disclosure provides a method of treating cancer in a human patient in need thereof, comprising administering to the patient a humanized monoclonal anti-human ILT2 IgG₄ antibody whose heavy chain CDR1-3 and light chain CDR1-3 comprise SEQ ID NOs: 1-6, respectively, in two or more treatment cycles, wherein each treatment cycle comprises a single dose of the anti-ILT2 antibody at 0.1-20 mg/kg per dose. In certain embodiments, the dose is 0.1, 0.3, 1, 3, 10, or 20 mg/kg. In certain embodiments, each treatment cycle is about two or three weeks. The antibody may, for example, comprise a heavy chain variable domain and a light chain variable domain comprising SEQ ID NOs: 7 and 8, respectively, or may comprise a heavy chain comprising SEQ ID NO: 9 (or SED ID NO: 9 without the C-terminal lysine) and a light chain comprising SEQ ID NO: 10.

In certain embodiments, the cancer is selected from the group consisting of breast cancer, triple negative breast cancer, biliary tract cancer, cervical cancer, cholangiocarcinoma, colorectal cancer, esophageal cancer, gastric cancer, gastroesophageal junction adenocarcinoma, hepatobiliary cancer, hepatocellular carcinoma, head and neck cancer, non-small cell lung cancer, renal cell carcinoma, skin squamous cell carcinoma, ovarian cancer, pancreatic cancer, renal cell carcinoma, and urothelial cancer.

In some embodiments, the antibody is administered at a first dose and a second dose. The first dose may be higher than the second dose, or the second dose may be higher than the first dose. For example,
the first dose may be 20 mg/kg, and the second dose may be 1 mg/kg;
the first dose may be 10 mg/kg, and the second dose may be 1 mg/kg;
the first dose may be 3 mg/kg, and the second dose may be 1 mg/kg;
the first dose may be 1 mg/kg, and the second dose may be 0.3 mg/kg; or
the first dose may be 0.3 mg/kg, and the second dose may be 0.1 mg/kg.

In some embodiments, the antibody is administered to the patient by intravenous infusion.

In some embodiments, the method further comprise administration of a single dose of pembrolizumab to the patient in each treatment cycle. For example, the pembrolizumab may be administered intravenously at 200 mg per dose. In certain embodiments, chemotherapy is also administered to the patient, e.g., carboplatin, pemetrexed, or both. In particular embodiments, the carboplatin may be administered intravenously to an AUC of 5 per treatment cycle. In particular embodiments, the pemetrexed may be administered intravenously at a dose of 500 mg/m² per treatment cycle. Optionally, the patient may be premedicated with folic acid, vitamin B12, dexamethasone, or any combination thereof before each administration of pemetrexed. In some embodiments, each treatment cycle is about three weeks.

In some embodiments, the method further comprises administration of a single dose of cetuximab to the patient in each treatment cycle. For example, the cetuximab may be administered intravenously at 500 mg/m² per dose. Optionally, the patient may be premedicated with a histamine-1 (H¹) receptor antagonist before each administration of cetuximab. In certain embodiments, the Hi receptor antagonist is diphenhydramine, which may be administered, e.g., at a dose of 50 mg. In some embodiments, each treatment cycle is about two weeks.

In some embodiments, the methods described herein are first line therapy, second line or later therapy, or third line or later therapy.

In some embodiments, the present disclosure provides a method of treating breast cancer, biliary tract cancer, cervical cancer, colorectal cancer, esophageal cancer, gastric cancer, hepatocellular carcinoma, head and neck cancer, non-small cell lung cancer, renal cell carcinoma, skin squamous cell carcinoma, or urothelial cancer in a human patient in need thereof, comprising administering to the patient by intravenous infusion a humanized monoclonal anti-human ILT2 antibody that comprises a heavy chain and a light chain having the amino acid sequences of SEQ ID NOs: 9 and 10, respectively, in two or more treatment cycles, wherein each treatment cycle comprises a single dose of the anti-ILT2 antibody at 0.1, 0.3, 1, 3, 10 or 20 mg/kg per dose, wherein each treatment cycle is about two weeks.

In some embodiments, the present disclosure provides a method of treating biliary tract cancer, cervical cancer, colorectal cancer, esophageal cancer, gastric cancer, hepatocellular carcinoma, head and neck cancer, non-small cell lung cancer, pancreatic cancer, renal cell carcinoma, skin squamous cell carcinoma, triple negative breast cancer, or urothelial cancer in a human patient in need thereof, comprising intravenously administering to the patient
a humanized monoclonal anti-human ILT2 antibody that comprises a heavy chain and
a light chain having the amino acid sequences of SEQ ID NOs: 9 and 10, respectively, and pembrolizumab,
   in two or more treatment cycles, wherein each treatment cycle comprises a single dose of the anti-ILT2 antibody at 0.1, 0.3, 1, 3, 10 or 20 mg/kg per dose, and a single 200 mg dose of pembrolizumab, wherein each treatment cycle is about three weeks.

In some embodiments, the present disclosure provides a method of treating colorectal cancer, head and neck cancer, non-small cell lung cancer, ovarian cancer, or hepatobiliary cancer in a human patient in need thereof, comprising intravenously administering to the patient
a humanized monoclonal anti-human ILT2 antibody that comprises a heavy chain and a light chain having the amino acid sequences of SEQ ID NOs: 9 and 10, respectively, and cetuximab,
in two or more treatment cycles, wherein each treatment cycle comprises a single dose of the anti-ILT2 antibody at 0.1, 0.3, 1, 3, 10 or 20 mg/kg per dose, and a single 500 mg/m² dose of cetuximab, wherein each treatment cycle is about two weeks. In certain embodiments, the patient may be premedicated with diphenhydramine before each administration of cetuximab, e.g., wherein the diphenhydramine is administered at a dose of 50 mg.

In any of the methods described herein, the humanized monoclonal anti-human ILT2 antibody may be administered at a first dose and a second dose. In some embodiments, the first dose is lower than the second dose.

In some embodiments, the present disclosure provides a method of treating cancer in a human patient in need thereof, comprising administering to the patient by intravenous infusion a humanized monoclonal anti-human ILT2 antibody that comprises a heavy chain and a light chain having the amino acid sequences of SEQ ID NOs: 9 and 10, respectively, in two or more treatment cycles, wherein each treatment cycle comprises a single dose of the anti-ILT2 antibody at 1, 3, 10 or 20 mg/kg per dose, wherein the cancer is advanced stage squamous cell carcinoma of the head and neck, gastric or gastroesophageal junction adenocarcinoma, cholangiocarcinoma, or non-small cell lung cancer (e.g., non-squamous non-small cell lung cancer), wherein each treatment cycle is about two weeks. In some embodiments, the present disclosure provides a method of treating non-squamous non-small cell lung cancer in a human patient in need thereof, comprising administering to the patient by intravenous infusion
a humanized monoclonal anti-human ILT2 antibody that comprises a heavy chain and a light chain having the amino acid sequences of SEQ ID NOs: 9 and 10, respectively,
pembrolizumab,
carboplatin, and
pemetrexed,
   in two or more treatment cycles, wherein each treatment cycle comprises
a single dose of the anti-ILT2 antibody at 1, 3, 10 or 20 mg/kg per dose,
a single 200 mg dose of pembrolizumab,
a single dose of carboplatin to AUC of 5, and
a single 500 mg/m2 dose of pemetrexed,
   wherein each treatment cycle is about three weeks, and optionally wherein the treatment is first line therapy. In certain embodiments, the patient is pretreated with folic acid, vitamin B12, dexamethasone, or any combination thereof before each administration of pemetrexed

In some embodiments, the present disclosure provides a method of treating non-small cell lung cancer in a human patient in need thereof, comprising intravenously administering to the patient
a humanized monoclonal anti-human ILT2 antibody that comprises a heavy chain and a light chain having the amino acid sequences of SEQ ID NOs: 9 and 10, respectively, and
pembrolizumab,
   in two or more treatment cycles, wherein each treatment cycle comprises a single dose of the anti-ILT2 antibody at 1, 3, 10 or 20 mg/kg per dose, and a single 200 mg dose of pembrolizumab,
   wherein each treatment cycle is about three weeks, and optionally wherein the treatment is second or third line therapy.

In some embodiments, the present disclosure provides a method of treating gastric cancer or gastroesophageal junction adenocarcinoma in a human patient in need thereof, comprising intravenously administering to the patient
a humanized monoclonal anti-human ILT2 antibody that comprises a heavy chain and a light chain having the amino acid sequences of SEQ ID NOs: 9 and 10, respectively, and pembrolizumab,
in two or more treatment cycles, wherein each treatment cycle comprises a single dose of the anti-ILT2 antibody at 1, 3, 10 or 20 mg/kg per dose, and a single 200 mg dose of pembrolizumab,
   wherein each treatment cycle is about three weeks, and optionally wherein the treatment is second line or later therapy or third line or later therapy. In certain embodiments, the treatment is second line or later therapy.

In some embodiments, the present disclosure provides a method of treating colorectal cancer in a human patient in need thereof, comprising intravenously administering to the patient
a humanized monoclonal anti-human ILT2 antibody that comprises a heavy chain and a light chain having the amino acid sequences of SEQ ID NOs: 9 and 10, respectively, and
cetuximab,
   in two or more treatment cycles, wherein each treatment cycle comprises a single dose of the anti-ILT2 antibody at 1, 3, 10 or 20 mg/kg per dose, and a single 500 mg/m² dose of cetuximab,
   wherein each treatment cycle is about two weeks, and optionally wherein the treatment is second line or later therapy or third line or later therapy. In certain embodiments, the treatment is second line or later therapy. In certain embodiments, the patient may be pretreated with diphenhydramine at a dose of 50 mg before each administration of cetuximab.

In some embodiments, the present disclosure provides a method of treating non-small cell lung cancer in a human patient in need thereof, comprising intravenously administering to the patient
a humanized monoclonal anti-human ILT2 antibody that comprises a heavy chain and a light chain having the amino acid sequences of SEQ ID NOs: 9 and 10, respectively, and
cetuximab,
   in two or more treatment cycles, wherein each treatment cycle comprises a single dose of the anti-ILT2 antibody at 1, 3, 10 or 20 mg/kg per dose, and a single 500 mg/m² dose of cetuximab,
   wherein each treatment cycle is about two weeks, and optionally wherein the treatment is second line or later therapy.

In some embodiments, the present disclosure provides a method of treating cholangiocarcinoma in a human patient in need thereof, comprising intravenously administering to the patient a humanized monoclonal anti-human ILT2 antibody that comprises a heavy chain and a light chain having the amino acid sequences of SEQ ID NOS: 9 and 10, respectively, in two or more treatment cycles, wherein each treatment cycle comprises a single dose of the anti-ILT2 antibody at 1, 3, 10 or 20 mg/kg per dose, wherein each treatment cycle is about two weeks, and optionally wherein the treatment is second line or later therapy.

In certain embodiments of the above-described methods, the single dose of the anti-ILT2 antibody is 3 mg/kg per dose.

In certain embodiments of the above-described methods, the single dose of the anti-ILT2 antibody is 10 mg/kg per dose.

In certain embodiments of the above-described methods, the single dose of the anti-ILT2 antibody is 20 mg/kg per dose.

In some embodiments, the methods described herein may be used to treat cancers that are unresectable or metastatic, to treat a patient who is refractory to standard approved therapy or who is not a candidate for standard approved therapy, or any combination thereof.

The present disclosure also provides an anti-human ILT2 antibody recited herein (optionally in combination with other agents described herein), for use in treating a human patient in a therapy recited herein, and use of a monoclonal anti-human ILT2 antibody recited herein (optionally in combination with other agents described herein) for the manufacture of a medicament for treating a human patient in a therapy recited herein. Kits and articles of manufacture comprising the recited anti-human ILT2 antibodies, optionally in combination with other agents described herein (e.g., for use in a therapy recited herein), are also provided.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIG. 1 is a line graph showing estimated average SAR444881 blood scrum concentrations in patients treated with one of the five recited doses of SAR444881 delivered intravenously at 14-day intervals. Error bars represent standard deviation.

FIG. 2 is a set of line graphs showing mean target ILT2 receptor occupancy (“RO”) by SAR444881 on circulating CD14+ monocytes during SAR444881 treatment, shown as a mean of all subjects treated in each dose-level cohort, with arrows marking SAR444881 infusion and error bars representing standard deviation. Panel A: receptor occupancy of SAR444881 in fresh whole blood samples in Sub-Part 1A patients (SAR444881). Panel B: receptor occupancy in Sub-Part (“Cohort”) 1C patients (SAR444881+cetuximab). Panel C: receptor occupancy in Sub-Part (“Cohort”) 1B patients (SAR444881+pembrolizumab).

FIG. 3 is a set of histograms indicating the percentage changes from baseline in mean fluorescence intensity (MFI) of CD62L on circulating classical CD14⁺CD16⁻ monocytes after SAR444881 treatment (Panel A); the fold changes from baseline of CD107a-expressing CD8⁺ T_{EMRA} cells after SAR444881 treatment (Panel B); and the fold changes from baseline of CD69-expressing HLA-DR⁺ILT2⁺natural killer cells after SAR444881 treatment (Panel C). Each symbol represents a patient at each dose level. Y-axis gray background represents a threshold of change of 15%. Circles depict patients with progressive disease (PD), triangles depict patients with stable disease (SD), and squares depict patients with a partial response (PR). TEMRA: effector memory T cells re-expressing CD45RA.

FIG. 4 is a set of histograms showing the percentage of ILT2-positive natural killer T (NKT) cells in the overall lymphocyte population (Panel A), the fold-change in Ki67-positive CD33 positive cells from baseline (Panel B), and the percentage-change in regulatory T (Treg) cells from baseline (Panel C), in response to SAR444881 treatment. Statistical significance was calculated using the Mann-Whitney non-parametric T-test for Panels A and B and the unpaired T-test for Panel C. Each symbol represents a patient and lines represent the median for each group. Circles depict patients with PD, triangles depict patients with SD, and squares depict patients with PR.

FIG. 5 is a set of graphs showing the maximum percent change from baseline in levels of serum CXCL11 during days 2-14 of SAR444881 treatment (Panel A), levels of serum CCL4 during days 14-28 of SAR444881 treatment (Panel B), and levels of serum CCL23 during cycle 2 of SAR444881 treatment (Panel C). Each symbol represents a subject at each dose level cohort.

FIG. 6 is a Kaplan-Meier plot showing the progression-free survival rate by dose following treatment with SAR444881 delivered intravenously at 14-day intervals.

### DETAILED DESCRIPTION OF THE INVENTION

The present disclosure provides safe and efficacious cancer treatment with SAR444881 or a related antibody (such as an antibody described herein, e.g., a humanized antibody having the same heavy and light chain CDRs or the same heavy and light chain variable domains as SAR444881). SAR444881 is a humanized monoclonal IgG₄ antibody targeting human ILT2. See also PCT Patent Publications WO 2021/028921 and WO 2022/034524

### I. Anti-ILT2 Antibodies

In some embodiments, a cancer therapy described herein uses an anti-ILT2 antibody that is SAR444881 or a related antibody (e.g., an antibody described herein), or an antigen-binding portion of said anti-ILT2 antibody. The SAR444881 heavy chain sequence (SEQ ID NO: 9) is shown below, with its variable domain sequence in boldface and italics (SEQ ID NO: 7) and its CDR1-3 (SEQ ID NOs: 1-3, respectively) underlined:

(SEQ ID NO: 9)
DVQLQGSGPG LVKPSETLSL TCSVTGYSIT SGYYWNWIRQ
FPGKKLEWMG YISYDGSNNY NPSLKNRITI SRDTSKNQFS
LKLNSVTAAD TATYYCAHGY SYYYAMDAWG QGTSVTVSSA
STKGPSVFPL APCSRSTSES TAALGCLVKD YFPEPVTVSW
NSGALTSGVH TFPAVLQSSG LYSLSSVVTV PSSSLGTKTY
TCNVDHKPSN TKVDKRVESK YGPPCPPCPA PEFEGGPSVF
LFPPKPKDTL MISRTPEVTC VVVDVSQEDP EVQFNWYVDG
VEVHNAKTKP REEQFNSTYR VVSVLTVLHQ DWLNGKEYKC
KVSNKGLPSS IEKTISKAKG QPREPQVYTL PPSQEEMTKN
QVSLTCLVKG FYPSDIAVEW ESNGQPENNY KTTPPVLDSD
GSFFLYSRLT VDKSRWQEGN VFSCSVMHEA LHNHYTQKSL
SLSLGK

The SAR444881 light chain sequence (SEQ ID NO: 10) is shown below, with its variable domain sequence in boldface and italics (SEQ ID NO: 8) and its CDR1-3 (SEQ ID NOs: 4-6, respectively) underlined:

(SEQ ID NO: 10)
DIQMTQSPSS LSASVGDRVT ITCRTSQDIS NYLNWYQQKP
GKAVKLLIS YTSRLHSGVPS RFSGSGSGTD YTLTISSLQP
EDFATYYCQQ GNTLPTFGQG TKLEIKRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSENR GEC

In some embodiments, the anti-ILT2 antibody is of human IgG₄ isotype subtype, optionally comprising mutations S228P and/or L235E (Eu numbering). In certain embodiments, (e.g., any of the embodiments described herein), the anti-ILT2 antibody is of isotype IgG₄ and has mutations S228P and L235E (Eu numbering).

In certain embodiments, the anti-ILT2 antibody comprises the six CDR amino acid sequences of SAR444881. The CDRs may be assigned in accordance with any method known in the art, such as IMGT® definitions (Lefranc et al., Dev Comp Immunol. (2003) 27(1): 55-77); or in accordance with the definitions of Kabat, Sequences of Proteins of Immunological Interest (National Institutes of Health, Bethesda, MD (1987 and 1991)); Chothia & Lesk, J Mol Biol. (1987) 196:901-17; Chothia et al., Nature (1989) 342:878-83; MacCallum et al., J Mol Biol. (1996) 262:732-45; or Honegger and Plückthun, J Mol Biol. (2001) 309 (3): 657-70, or any combination of these definitions (Kabat plus Chothia, for example). Examples of CDR definitions under different methods is shown below for SAR444881 (SEQ: SEQ ID NO):

SAR444881 HCDRs

| Definition | HCDR1 | SEQ | HCDR2 | SEQ | HCDR3 | SEQ |
|---|---|---|---|---|---|---|
| Kabat | SGYYWN | 1 | YISYDGSN | 2 | GYSYYYAMDA | 3 |
| | | | NYNPSLKN | | | |
| Chothia | GYSITSGY | 11 | SYDGS | 12 | GYSYYYAMDA | 3 |
| IMGT® | GYSITSGYY | 13 | ISYDGSN | 14 | AHGYSYYYAM | 15 |
| | | | | | DA | |
| Contact | TSGYYWN | 16 | WMGYISYD | 17 | AHGYSYYYAM | 18 |
| | | | GSNN | | D | |

SAR444881 LCDRs

| Definition | LCDR1 | SEQ | LCDR2 | SEQ | LCDR3 | SEQ |
|---|---|---|---|---|---|---|
| Kabat | RTSQDI | 4 | YTSRLHS | 5 | QQGNT | 6 |
| | SNYLN | | | | LPT | |
| Chothia | RTSQDI | 4 | YTSRLHS | 5 | QQGNT | 6 |
| | SNYLN | | | | LPT | |
| IMGT® | QDISNY | 19 | YTS | 20 | QQGNT | 6 |
| | | | | | LPT | |
| Contact | SNYLNWY | 21 | LLISYTS | 22 | QQGNT | 23 |
| | | | RLH | | LP | |

Thus, for example, the SAR444881 IMGT®-defined HCDR1-3 and LCDR1-3 sequences of
SEQ ID NOs: 1-6, respectively, may be replaced in any embodiment described herein by
SEQ ID NOs: 11, 12, 3, 4, 5, and 6, respectively;
SEQ ID NOs: 13, 14, 15, 19, 20, and 6, respectively; or
SEQ ID NOs: 16, 17, 18, 21, 22, and 23, respectively.
Also contemplated is a set of SAR444881 CDRs wherein each of HCDR1, HCDR2, HCDR3,LCDR1, LCDR2, and LCDR3 may individually be specified according to any of the methods for defining SAR444881 CDRs as shown above (e.g., HCDR1 specified by the Kabat definition, HCDR2 specified by the Chothia definition, etc.).

In certain embodiments, the anti-ILT2 antibody comprises HCDR1-3 and LCDR1-3 amino acid sequences of SEQ ID NOs: 1-6, respectively.

In some embodiments, the anti-ILT2 antibody comprises the heavy and light chain variable domain amino acid sequences of SAR444881. In certain embodiments, the anti-ILT2 antibody may comprise a heavy chain variable domain (V_{H}) that is at least 90% (e.g., at least 95%, 98%, or 99%) identical in sequence to SEQ ID NO: 7, and a light chain variable domain (V_{L}) that is at least 90% (e.g., at least 95%, 98%, or 99%) identical in sequence to SEQ ID NO: 8. Optionally, said antibody may comprise the six CDRs of SAR444881 (e.g., SEQ ID NOs: 1-6). In certain embodiments, the anti-ILT2 antibody comprises a V_{H} comprising SEQ ID NO: 7 and a VI. comprising SEQ ID NO: 8.

In some embodiments, the anti-ILT2 antibody comprises the heavy and light chain amino acid sequences of SAR444881. In certain embodiments, the anti-ILT2 antibody comprises a heavy chain (HC) that is at least 90% (e.g., at least 95%, 98%, or 99%) identical in sequence to SEQ ID NO: 9, and a light chain (LC) that is at least 90% (e.g., at least 95%, 98%, or 99%) identical in sequence to SEQ ID NO: 10. Optionally, said antibody may comprise the six CDRs (e.g., SEQ ID NOs: 1-6) or the V_{H} and V_{L}. (e.g., SEQ ID NOs: 7 and 8, respectively) of SAR444881. In certain embodiments, the anti-ILT2 antibody comprises an HC comprising SEQ ID NO: 9 (optionally without the C-terminal lysine) and an LC comprising SEQ ID NO: 10.

SAR444881 can be provided, for example, in solid form (e.g., lyophilized form) that is reconstituted in a suitable pharmaceutical solution before administration to a patient, or in an aqueous pharmaceutical solution. In certain embodiments, the antibody is provided in a pharmaceutical composition that further comprises a pharmaceutically acceptable excipient.

In some embodiments, a pharmaceutical composition comprising an anti-ILT2 antibody as recited herein (e.g., SAR444881) is provided in an article of manufacture or kit such as one that comprises one or more containers containing the composition and a label associated with the container(s). The container may be a single use container (e.g., for intravenous delivery), such as a single use boule or vial, or a single use, pre-filled syringe or injector. In some embodiments, the container contains a single dose (e.g., a dose recited herein) of the anti-ILT2 antibody, wherein the container may be a vial or a pre-filled syringe or injector. In some embodiments, the article of manufacture or kit further comprises one or more containers comprising additional agents, e.g., those administered in addition to SAR444881 in the combination therapies described herein. For example, the one or more containers may comprise pembrolizumab, cetuximab, carboplatin, pemetrexed, or any combination thereof, e.g., in single or multiple doses described herein for those agents. In certain embodiments, an article of manufacture or kit of the present disclosure comprises 1) containers comprising SAR444881; 2) containers comprising SAR444881 and pembrolizumab; 3) containers comprising SAR444881, pembrolizumab, pemetrexed, and carboplatin; or 4) containers comprising SAR444881 and cetuximab; optionally wherein the containers are for intravenous delivery.

### II. Patient Populations

The present disclosure relates to treatment of cancer in human patients in need thereof with SAR444881 or a related anti-ILT2 antibody, such as an anti-ILT2 antibody described herein. The treatment may further comprise pembrolizumab (e.g., in combination with chemotherapy such as carboplatin and/or pemetrexed) or cetuximab.

In some embodiments, the patient is an adult (≥18 years of age).

In some embodiments, the patient has unresectable or metastatic disease and is refractory to or is not a candidate for standard approved therapy. In some embodiments, the patient has failed all standard of care therapies. In some embodiments, the patient has received and failed prior treatment with pembrolizumab or another anti-PD-1 or anti-PD-L1 therapy. In some embodiments, the patient has received and failed prior treatment with fluoropyrimidine, oxaliplatin, or irinotecan, with bevacizumab and/or cetuximab. In some embodiments, the patient has received and failed prior treatment with fluoropyrimidine, oxaliplatin, irinotecan, bevacizumab and/or cetuximab. In some embodiments (e.g., where the patient has RAS wild-type colorectal cancer), the patient has received and failed, or relapsed on, or is ineligible for treatment with, 5-fluorouracil, irinotecan, oxaliplatin, bevacizumab and anti-EGFR. In some embodiments (e.g., where the patient has RAS-mutant colorectal cancer), the patient has received and failed, or relapsed on, or is ineligible for treatment with, bevacizumab. In some embodiments (e.g., where the patient has BRAF-mutant colorectal cancer), the patient has received and failed, or relapsed on, or is ineligible for treatment with, encorafenib or other BRAF targeted therapies.

In some embodiments, the patient has Eastern Cooperative Oncology Group Performance Status (ECOG) of 0 or 1.

In some embodiments, the patient has been diagnosed with one or more conditions selected from the group consisting of: breast cancer (e.g., triple negative breast cancer), cervical cancer, colorectal cancer (e.g., K-Ras wild-type colorectal cancer or BRAF-mutant colorectal cancer; optionally non-MSI-H disease), adenocarcinoma or squamous cell carcinoma of the esophagus, gastric or gastroesophageal junction adenocarcinoma (e.g., Siewert Types 2 and 3), pancreatic adenocarcinoma, squamous cell carcinoma of the head and neck or the skin, hepatobiliary cancers (e.g., hepatocellular carcinoma, gallbladder cancer, or cholangiocarcinoma, such as advanced cholangiocarcinoma), non-small cell lung cancer (e.g., stage IV squamous or non-squamous NSCLC as per American Joint Committee on Cancer [AJCC] 8^{th} edition), renal cell carcinoma, or urothelial carcinoma.

In some embodiments, the patient does not have active, known, or suspected autoimmune disease (optionally except for one or more of type I diabetes mellitus, hypothyroidism only requiring hormone replacement, a skin disorder not requiring systemic treatment (such as vitiligo, psoriasis, or alopecia), or a condition not expected to recur in the absence of an external trigger).

In some embodiments, the patient does not have known active CNS metastases and/or carcinomatous meningitis.

In some embodiments, the patient does not have coronary artery disease or a history of myocardial infarction, high risk of uncontrolled arrhythmia or uncontrolled cardiac insufficiency, and/or uncontrolled or poorly controlled hypertension (e.g., >180 mmHg systolic or >130 mmHg diastolic).

In some embodiments, the patient demonstrates increased baseline levels of ILT2-positive immune cells (e.g., NKT cells, PD-1-negative CD8 T_{EMRA} cells, and/or dendritic cells), and/or increased levels of soluble HLA-G prior to treatment. Levels of ILT2-positive immune cells and/or soluble HLA-G may be increased from baseline by, e.g., by at least 10%, 20%, 30%, 40%, 50%, 60%, 70%, 80%, 90%, 100%, 200%, 300%, 400%, or 500%.

In some embodiments, the patient fulfills one or more of the inclusion criteria listed in Examples 1 and 2 below. In certain embodiments, the patient fulfills all of the inclusion criteria listed in Example 1 for a given indication or dosage regimen. In certain embodiments, the patient fulfills all of the inclusion criteria listed in Example 2 for a given indication or dosage regimen.

In some embodiments, the patient does not fulfill one or more of the exclusion criteria listed in Examples 1 and 2 below. In certain embodiments, the patient does not fulfill any of the exclusion criteria listed in Example 1 for a given indication or dosage regimen. In certain embodiments, the patient does not fulfill any of the exclusion criteria listed in Example 2 for a given indication or dosage regimen.

### III. Treatment of Cancer

The present disclosure relates to treating cancer in a patient as described herein, with SAR444881 or a related anti-ILT2 antibody (e.g., an anti-ILT2 antibody described herein). In some embodiments, the treatment further comprises pembrolizumab (e.g., in combination with chemotherapy such as carboplatin and/or pemetrexed) or cetuximab.

The present therapies can be used as a first line therapy to treat treatment-naïve patients, i.e., those who have not been treated with anti-cancer drugs (e.g., drugs against the specific cancer with which the patient presents). The present therapies can also be used to treat patients who have been treated with anti-cancer drugs (e.g., drugs against the specific cancer with which the patient presents), but these patients may have failed to respond to the previous treatment, or may have since experienced worsening of the disease or renewed disease activity. In some embodiments, the present therapies are used as a second line or later therapy. In some embodiments, the present therapies are used as a third line or later therapy.

In some embodiments, the present therapies are used to treat a cancer that is unresectable. In some embodiments, the present therapies are used to treat a cancer that is metastatic. In some embodiments, the patient is refractory to standard approved therapy. In some embodiments, the patient is not a candidate for standard approved therapy. In some embodiments, the patient has failed all standard approved therapies. Any combination of the above is also contemplated.

Administration of the anti-ILT2 antibody may be parenteral, e.g., intravenous. For example, the anti-ILT2 antibody may be administered through an intravenous infusion drip over about 15, 30, 45, 60, 75, or 90 minutes. In some embodiments, the antibody is intravenously administered at a dose of 0.05-50 mg/kg, such as at a dose of 0.1-20 mg/kg, e.g., about 0.1, 0.3, 0.5, 1, 1.5, 3, 5, 10, 15, or 20 mg/kg, per treatment cycle. In certain embodiments, the dose is 0.1, 0.3, 1, 3, 10, or 20 mg/kg per treatment cycle. In particular embodiments, the dose is 1, 3, 10, or 20 mg/kg per treatment cycle. In some embodiments, each treatment cycle is about 1, 2, 3, 4, 5, 6, 7, or 8 weeks. In certain embodiments, each treatment cycle is about two weeks or about three weeks. The therapy may comprise 1, 2, 3, 4, 5, 6, 7, 8, 9, 10, 15, 20, or more treatment cycles (optionally consecutive). In particular embodiments, the therapy comprises two or more treatment cycles (optionally consecutive). Where treatment cycles are consecutive, there is no period of delay between cycles; for example, for consecutive two week treatment cycles, the day after day 14 of one treatment cycle would be day 1 of the next treatment cycle.

In certain embodiments, the antibody is administered through an intravenous

infusion drip over about 30 minutes (e.g., at a dose of 0.1 mg/kg). In some embodiments, the antibody is administered through an intravenous infusion drip over about 60 minutes (e.g., at a dose of 0.3, 1, 3, 10, or 20 mg/kg).

In some embodiments, the anti-ILT2 antibody is intravenously administered at a dose of 1 mg/kg every two weeks.

In some embodiments, the anti-ILT2 antibody is intravenously administered at a dose of 3 mg/kg every two weeks.

In some embodiments, the anti-ILT2 antibody is intravenously administered at a dose of 10 mg/kg every two weeks.

In some embodiments, the anti-ILT2 antibody is intravenously administered at a dose of 20 mg/kg every two weeks.

In some embodiments, the anti-ILT2 antibody is intravenously administered at a dose of 1 mg/kg every three weeks.

In some embodiments, the anti-ILT2 antibody is intravenously administered at a dose of 3 mg/kg every three weeks.

In some embodiments, the anti-ILT2 antibody is intravenously administered at a dose of 10 mg/kg every three weeks.

In some embodiments, the anti-ILT2 antibody is intravenously administered at a dose of 20 mg/kg every three weeks.

In some embodiments, the anti-ILT2 antibody may be intravenously administered to the patient at two or more doses (e.g., doses recited above) in different treatment cycles. For example, the first administration of the antibody may be at a first dose and subsequent administration of the antibody may be at a second dose. In certain embodiments, the first dose is lower than the second dose. In certain embodiments, the first dose (“loading dose”) is higher than the second dose. For example, the first and second antibody doses may be:
a) 20 mg/kg and 1 mg/kg;
b) 10 mg/kg and 1 mg/kg;
c) 3 mg/kg, and 1 mg/kg;
d) 1 mg/kg and 0.3 mg/kg; or
e) 0.3 mg/kg and 0.1 mg/kg

In certain embodiments, the anti-ILT2 antibody is used to treat a cancer, e.g., a PD-1-related or PD-L1-related cancer. A PD-1-related cancer may be, e.g., a cancer associated with the binding of PD-1 to PD-L1 and/or PD-L2, and may in some embodiments to be associated with elevated expression of PD-1 (e.g., as determined in a tumor sample from a patient). A PD-L1-related cancer may be, e.g., a cancer associated with the binding of PD-L1 to PD-1, and may in some embodiments be associated with elevated expression of PD-L1 (e.g., as determined in a tumor sample from a patient). In particular embodiments, the cancer is selected from breast cancer (e.g., triple negative breast cancer), biliary tract cancer, cervical cancer, cholangiocarcinoma, colorectal cancer, esophageal cancer (e.g., adenocarcinoma or squamous cell carcinoma of the esophagus), gastric cancer, gastroesophageal junction adenocarcinoma, hepatobiliary cancer, hepatocellular carcinoma, head and neck cancer (e.g., squamous cell carcinoma of the head and neck), non-small cell lung cancer (e.g., squamous or non-squamous non-small cell lung cancer), renal cell carcinoma, skin squamous cell carcinoma, ovarian cancer, pancreatic cancer, renal cell carcinoma, and urothelial cancer.

### Anti-ILT2 Antibody Monotherapy

In some embodiments, an anti-ILT2 antibody described herein is administered alone (monotherapy). In certain embodiments, the anti-ILT2 antibody monotherapy is used to treat a cancer selected from breast cancer, biliary tract cancer, cervical cancer, colorectal cancer, esophageal cancer, gastric cancer, hepatocellular carcinoma, head and neck cancer, non-small cell lung cancer, renal cell carcinoma, skin squamous cell carcinoma, and urothelial cancer. In certain embodiments, the anti-ILT2 antibody monotherapy is used to treat a cancer selected from advanced stage squamous cell carcinoma of the head and neck, gastric or gastroesophageal junction adenocarcinoma, cholangiocarcinoma, or non-squamous non-small cell lung cancer. In particular embodiments, the anti-ILT2 antibody monotherapy is used to treat cholangiocarcinoma. In some embodiments, the treatment may comprise SAR444881 at a dose of 1, 3, 10, or 20 mg/kg per treatment cycle. In certain embodiments, each treatment cycle is about two weeks.

### Anti-ILT2 Antibody and Pembrolizumab

In some embodiments, the anti-ILT2 antibody described herein (e.g., SAR444881) is administered in combination with an anti-PD-1 or anti-PD-LI agent, e.g., an anti-PD-1 antibody such as pembrolizumab. In certain embodiments, the pembrolizumab is administered intravenously to the patient (such as through intravenous infusion drip over, for example, about 30 minutes), for example at a dose of 100, 150, 200, 250, or 300 mg per treatment cycle. In particular embodiments, the pembrolizumab is administered intravenously to the patient at a dose of 200 mg per treatment cycle.

In some embodiments, the combination therapy comprising an anti-ILT2 antibody described herein (e.g., SAR444881) and an anti-PD-1 or anti-PD-LI agent (e.g., pembrolizumab) further comprises one or more chemotherapeutic agents (e.g., carboplatin and/or pemetrexed). In certain embodiments, the combination therapy comprises SAR444881, pembrolizumab, carboplatin, and pemetrexed. In particular embodiments, the carboplatin is administered intravenously (such as through intravenous infusion drip) in the combination therapy to an AUC (area under the concentration-time curve) of 2.5, 3.75, or 5 (e.g., to an AUC of 5) per treatment cycle. In some embodiments, carboplatin is administered for no more than 3, 4, or 5 (e.g., 4) treatment cycles. In particular embodiments, the pemetrexed is administered intravenously (such as through intravenous infusion drip, for example, over about 10 minutes) at a dose of 250, 375, or 500 mg/m² (e.g., 500 mg/m²) per treatment cycle. Optionally, one or more administrations of pemetrexed (e.g., all administrations) may be preceded by premedication with folic acid, vitamin B12, dexamethasone, or any combination thereof. In some embodiments, the folic acid is administered at an oral dose of about 350-1000 μg. For example, in certain embodiments, five daily doses of folic acid may be taken during the seven days preceding the first dose of pemetrexed, and folic acid dosing continues during the full course of therapy and for about 21 days after the last dose of pemetrexed. In some embodiments, the vitamin B12 is administered at a dose of about 1000 μg through intramuscular injection (e.g., in the week preceding the first dose of pemetrexed and once every three cycles thereafter, optionally on the same day as pemetrexed administration). In some embodiments, the dexamethasone is administered at an oral dose of about 4 mg twice per day (or equivalent), e.g., the day before, day of, and day after pemetrexed administration.

In some embodiments, the combination therapy comprising an anti-ILT2 antibody described herein (e.g., SAR444881), an anti-PD-1 or anti-PD-LI agent (e.g., pembrolizumab), and optionally one or more chemotherapeutic agents (e.g., carboplatin and/or pemetrexed) is administered in treatment cycles of about two, three, or four weeks. In certain embodiments, each treatment cycle is about three weeks.

In some embodiments, the combination therapy comprising an anti-ILT2 antibody described herein (e.g., SAR444881) and an anti-PD-1 or anti-PD-L1 agent (e.g., pembrolizumab) is used to treat a cancer selected from biliary tract cancer, cervical cancer, colorectal cancer, esophageal cancer, gastric cancer, hepatocellular carcinoma, head and neck cancer, non-small cell lung cancer, pancreatic cancer, renal cell carcinoma, skin squamous cell carcinoma, triple negative breast cancer, and urothelial cancer. For example, the combination therapy may comprise SAR444881 at a dose of 1, 3, 10, or 20 mg/kg per treatment cycle and pembrolizumab at a dose of 200 mg per treatment cycle. In certain embodiments, each treatment cycle is about three weeks.

In some embodiments, the combination therapy comprising an anti-ILT2 antibody described herein (e.g., SAR444881) and an anti-PD-1 or anti-PD-LI agent (e.g., pembrolizumab) is used to treat lung cancer, e.g., non-small cell lung cancer. For example, the combination therapy may comprise SAR444881 at a dose of 1, 3, 10, or 20 mg/kg per treatment cycle and pembrolizumab at a dose of 200 mg per treatment cycle. In certain embodiments, each treatment cycle is about three weeks. In certain embodiments, the combination therapy is second line or later therapy. In certain embodiments, the combination therapy is third line or later therapy.

In some embodiments, the combination therapy comprising an anti-ILT2 antibody described herein (e.g., SAR444881) and an anti-PD-1 or anti-PD-LI agent (e.g., pembrolizumab) is used to treat gastric cancer or gastroesophageal junction adenocarcinoma. For example, the combination therapy may comprise SAR444881 at a dose of 1, 3, 10, or 20 mg/kg per treatment cycle and pembrolizumab at a dose of 200 mg per treatment cycle. In certain embodiments, each treatment cycle is about three weeks. In certain embodiments, the combination therapy is second line or later therapy. In certain embodiments, the combination therapy is third line or later therapy.

In some embodiments, the combination therapy comprising an anti-ILT2 antibody described herein (e.g., SAR444881), an anti-PD-1 or anti-PD-LI agent (e.g., pembrolizumab), and optionally one or more chemotherapeutic agents (e.g., carboplatin and/or pemetrexed) is used to treat lung cancer, e.g., non-squamous non-small cell lung cancer. For example, the combination therapy may comprise SAR444881 at a dose of 1, 3, 10, or 20 mg/kg per treatment cycle, pembrolizumab at a dose of 200 mg per treatment cycle, carboplatin to a dose to AUC of 5 per treatment cycle, and pemetrexed at a dose of mg/m² per treatment cycle. In certain embodiments, each treatment cycle is about three weeks. In certain embodiments, the combination therapy is first line therapy. In certain embodiments, the combination therapy is second line or later therapy. In certain embodiments, the combination therapy is third line or later therapy. In particular embodiments, the patient is pretreated with folic acid, vitamin B12, and/or dexamethasone (e.g., all three) before each administration of pemetrexed. In some embodiments, carboplatin is administered for no more than 3, 4, or 5 (e.g., 4) treatment cycles.

### Anti-ILT2 Antibody and Cetuximab

In some embodiments, the anti-ILT2 antibody described herein (e.g., SAR444881) is administered in combination with an anti-EGFR agent, e.g., an anti-EGFR antibody such as cetuximab. In certain embodiments, the cetuximab is administered intravenously (such as through intravenous infusion drip, for example, over about 120 minutes) to the patient, e.g., at a dose of 400, 450, 500, 550, or 600 mg/m² per treatment cycle. In particular embodiments, the cetuximab is administered intravenously to the patient at a dose of 500 mg/m² per treatment cycle. Optionally, one or more administrations of cetuximab (e.g., all administrations) may be preceded by premedication with a histamine-1 (H₁) receptor antagonist (and additionally or alternatively with acetaminophen/paracetamol and/or corticosteroids). Administration of the H₁ receptor antagonist may be intravenous (such as through intravenous infusion drip, for example over about 30-60 minutes). In certain embodiments, the H₁ receptor antagonist is diphenhydramine. The diphenhydramine may be administered at a dose of, e.g., 30, 40, or 50 mg per treatment cycle, and in particular embodiments is administered at a dose of 50 mg per treatment cycle.

In some embodiments, the combination therapy comprising an anti-ILT2 antibody described herein (e.g., SAR444881) and an anti-EGFR agent (e.g., cetuximab) is administered in treatment cycles of about one, two, or three, weeks. In certain embodiments, each treatment cycle is about two weeks.

In some embodiments, the combination therapy comprising an anti-ILT2 antibody described herein (e.g., SAR444881) and an anti-EGFR agent (e.g., cetuximab) is used to treat a cancer selected from colorectal cancer, head and neck cancer, non-small cell lung cancer, ovarian cancer, and hepatobiliary cancer. In some embodiments, the hepatobiliary cancer is hepatocellular carcinoma, gallbladder cancer, or cholangiocarcinoma.

In some embodiments, the combination therapy comprising an anti-ILT2 antibody described herein (e.g., SAR444881) and an anti-EGFR agent (e.g., cetuximab) is used to treat colorectal cancer. For example, the combination therapy may comprise SAR444881 at a dose of 1, 3, 10, or 20 mg/kg per treatment cycle and cetuximab at a dose of 500 mg/m² per treatment cycle. In certain embodiments, each treatment cycle is about two weeks. In certain embodiments, the combination therapy is second line or later therapy. In certain embodiments, the combination therapy is third line or later therapy. Optionally, each administration of cetuximab may be preceded by premedication with diphenhydramine, e.g., at a dose of 50 mg per treatment cycle.

In some embodiments, the combination therapy comprising an anti-ILT2 antibody described herein (e.g., SAR444881) and an anti-EGFR agent (e.g., cetuximab) is used to treat non-small cell lung cancer. For example, the combination therapy may comprise SAR444881 at a dose of 1, 3, 10, or 20 mg/kg per treatment cycle and cetuximab at a dose of 500 mg/m² per treatment cycle. In certain embodiments, each treatment cycle is about two weeks. In certain embodiments, the combination therapy is second line or later therapy. In certain embodiments, the combination therapy is third line or later therapy. Optionally, each administration of cetuximab may be preceded by premedication with diphenhydramine, e.g., at a dose of 50 mg per treatment cycle.

As used herein, the term “treatment cycle” refers to a period during which agents that comprise a therapeutic regimen are administered on proscribed days; the treatment cycle may be repeated on a regular schedule. For example, in a two-week treatment cycle of SAR444881 monotherapy, SAR444881 may be administered on day 1 of each cycle (e.g., every two weeks). For combination therapies described herein, the recited doses of the agents may be administered on the same day of the treatment cycle, on different days of the treatment cycle, or any combination thereof for more than two agents. For example, SAR444881 may be administered on the same or different days of the treatment cycle from pembrolizumab or cetuximab. In embodiments where the combination therapy comprises SAR444881, pembrolizumab, and chemotherapy (e.g., carboplatin and/or pemetrexed), SAR444881 may be administered on the same day of the treatment cycle as pembrolizumab, but on a different day from the chemotherapy; on different days from pembrolizumab and the chemotherapy (which may be administered on the same or different days); or on the same day as the chemotherapy but on a different day from pembrolizumab. In certain embodiments, the recited doses of the agents are all administered on the same day of the treatment cycle. In some embodiments, pembrolizumab or cetuximab will be administered first, followed by SAR444881. In certain embodiments, SAR444881 administration starts at least about 30 minutes after completion of pembrolizumab administration. In certain embodiments, SAR444881 administration starts at least about 60 minutes after completion of cetuximab administration. For treatments including pembrolizumab and chemotherapy, in some embodiments, the treatment follows the following dosing sequence: pembrolizumab, chemotherapy, and SAR444881 (e.g., wherein the SAR444881 administration is at least about 30 minutes after completion of pembrolizumab administration). For example, where the chemotherapy comprises pemetrexed and carboplatin, the dosing sequence may be, e.g.: pembrolizumab, pemetrexed premedication, pemetrexed, carboplatin, and SAR444881.

In certain embodiments, doses recited herein for any or all agents in the described therapies are single doses per treatment cycle.

In some embodiments (e.g., in the event of a noted or expected infusion reaction), intravenous infusion(s) in a treatment recited herein may be preceded by a prophylactic premedication. In certain embodiments, the premedication may be diphenhydramine (e.g., at a dose of about 50 mg) and/or acetaminophen/paracetamol (e.g., at a dose of about 325-1000 mg). In some embodiments, the premedication is administered at least about 15, 30, or 45 (e.g., at least about 30) minutes before subsequent treatment infusions. In some embodiments, infusion reactions may also be treated or prevented using corticosteroids (e.g., up to about 25 mg of hydrocortisone sodium succinate or equivalent).

The present therapies are contemplated to be efficacious in cancer patients. The therapies may, e.g., result in inhibition of tumor growth, tumor regression, slowing or reversal of metastasis, prolonged survival, prolonged progression-free survival, prevention of cancer recurrence or residual disease, alleviation of cancer symptoms, or any combination thereof. In some embodiments, the therapies increase immune activity in the patient. The increased immune activity may be demonstrated, e.g., by increased levels of CD62L on the surface of classical monocytes. CD62L, also known as L-selectin, is a cell adhesion molecule that plays a role in regulating the recruitment of monocytes to tissues from the blood during inflammation. Additionally or alternatively, the increased immune activity may be demonstrated by increased levels of immune activation markers such as CD69 (e.g., on ILT2-expressing natural killer T cells) and CD107a (e.g., on ILT2-expressing CD8 TEMRA and natural killer cells).

In some embodiments, the present therapies increase expression of CD62L on classical monocytes in patients. In some embodiments, the present therapies increase the levels of cytokines such as CCL4, CXCL11, CCL23, granzyme B, TNFα, IFNγ, GM-CSF, or any combination thereof, in patients. In some embodiments, the present therapies downregulate CCL7 in patients. In some embodiments, the present therapies activate monocytes. In some embodiments, the present therapies activate ILT2-expressing T and/or NK cell subsets. In some embodiments, the present therapies increase intratumoral necrosis. In some embodiments, the present therapies increase tumor infiltration of CD8 T cells. Any combination of the above effects is also contemplated.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any treatment method disclosed herein may be used to treat any individual as defined herein. Further, headers herein are created for case of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Study of SAR444881 Administered Alone and in Combination with Other Therapeutics in Participants with Advanced Solid Tumors

This Example outlines the protocol and preliminary results for an open-label, multicenter, Phase 1/2, first-in-human study of the safety, tolerability, and anti-tumor activity of intravenous SAR444881, a humanized monoclonal IgG₄ antibody targeting ILT2. The study will evaluate SAR444881, as a single agent and when combined with pembrolizumab or with cetuximab, for treatment of cancer patients with advanced solid tumors where no other standard of care treatment option is available. Given the known mechanism of action of ILT2 and its potential role in cancer, the study focuses on tumor types known to express HLA-G. Tumor types included in SAR444881-cetuximab combination cohorts are tumors known to over-express the EGFR protein.

### Study Design

The proposed study is comprised of two parts-a dose escalation phase (Part 1) and a dose optimization/expansion phase (Part 2). Part 1 is comprised of three sub-parts: SAR444881 administered as a monotherapy (Sub-Part 1A), SAR444881 administered in combination with pembrolizumab (Sub-Part 1B), and SAR444881 administered in combination with cetuximab (Sub-Part 1C). Part 2 is composed of two sub-parts: a dose optimization part where up to two doses of SAR444881 per indication are administered in combination with pembrolizumab, pembrolizumab and chemotherapy, or cetuximab (Sub-Part 2A); and a dose expansion part where SAR444881 is administered alone (Sub-Part 2B).

The objectives for the dose escalation phase (Part 1) are to assess the safety and tolerability, and to determine the MTD (or MAAD, if no MTD is reached) and the recommended dose(s) of SAR444881 when administered alone and in combination with pembrolizumab or with cetuximab. Dose escalation in all three sub-parts will follow a standard “3+3” design enrolling at least 3 participants per dose level cohort. The dose escalation phase of the study will enroll cancer patients with advanced disease who are refractory to or are not candidates for standard approved therapy.

Part 2, which includes five cohorts, is designed to assess preliminary anti-tumor activity and optimal dose of SAR444881 as a single agent and in combination in cancer patients with select advanced solid tumors known to express HLA-G and a likelihood of sensitivity to immunotherapies as observed with other checkpoint inhibitors. It will enroll participants with unresectable or metastatic disease. The proposed study aims to establish proof-of-concept for SAR444881 as a monotherapy or in combination with: 1) the anti-PD1 monoclonal antibody pembrolizumab; 2) the anti-EGFR monoclonal antibody cetuximab; or 3) pembrolizumab and chemotherapy. Part 2 is divided into two sub-parts: in Sub-Part 2A (Dose Optimization), the preliminary anti-tumor activity and optimal dose of SAR444881 is assessed in combination with the aforementioned anti-tumor agents; and in Sub-Part 2B (Dose Expansion), the preliminary anti-tumor activity of SAR444881 is assessed as a monotherapy. An overview of the study cohorts is provided below:

| | Study | | |
|---|---|---|---|
| Cohort | Sub-Part | Study Intervention | Disease |
| A1 | 2A | SAR444881 + pembrolizumab + | Non-squamous |
| | | carboplatin + pemetrexed | NSCLC |
| | | as 1L therapy | |
| B1 | 2A | SAR444881 + pembrolizumab | GC/GEJ |
| | | as 2L and later therapy | |
| C1 | 2A | SAR444881 + cetuximab as | CRC any RAS |
| | | 2L and later therapy | |
| D1 | 2B | SAR444881 as 2L and later | Cholangio- |
| | | therapy | carcinoma |
| E1 | 2A | SAR444881 + cetuximab | NSCLC |
| | | as 2L and later therapy | |

1L = first line;
2L = second line;
NSCLC = non-small cell lung cancer;
GC/GEJ = gastric cancer or gastro-esophageal junction adenocarcinoma;
CRC = colorectal carcinoma.

### Objectives

The primary objectives of the dose escalation phase of this study (Part 1) are to assess the safety and tolerability of SAR444881, the MTD or MAAD, and the recommended doses of SAR444881 for expansion/optimization when administered alone or in combination with pembrolizumab or with cetuximab.

The secondary objectives of the dose escalation phase of this study are:
To assess preliminary anti-tumor activity of SAR444881 as a monotherapy and in combination with pembrolizumab or with cetuximab;
To characterize the pharmacokinetics (PK) of SAR444881 as a monotherapy and in combination with pembrolizumab or with cetuximab; and. To characterize the immunogenicity of SAR444881 as a monotherapy and in combination with pembrolizumab or with cetuximab.

The exploratory objectives of the dose escalation phase of this study are:
To explore potential associations between SAR444881 anti-tumor activity and select biomarker measures in the tumor and peripheral blood; and.
To explore the associations between SAR444881 serum PK, safety, efficacy, and clinical biomarkers.

The primary objectives of the dose expansion/optimization phase of this study (Part 2) are to assess the preliminary antitumor activity of SAR444881 (alone or in combination with pembrolizumab, cetuximab, or pembrolizumab and chemotherapy), and to define the optimal dose of SAR444881 in combination with pembrolizumab, cetuximab, or pembrolizumab and chemotherapy.

The secondary objectives of the dose expansion/optimization phase of this study are:
To assess additional parameters of anti-tumor activity of SAR444881;
To further assess the safety and tolerability of SAR444881;
To further characterize the PK of SAR444881 and pembrolizumab or cetuximab (in combination only for dose optimization/expansion); and.
To further characterize the immunogenicity of SAR444881.

The exploratory objectives of the dose expansion/optimization phase of this study are:
To explore the potential associations between SAR444881 anti-tumor activity and select biomarker measures in the tumor and peripheral blood;
To explore the associations between SAR444881 serum PK, safety, efficacy, and clinical biomarkers; and
To explore the effect of SAR444881 on patient-reported outcomes (PROs).

### Endpoints

### Dose Escalation (Part 1)

The primary endpoints for the dose escalation phase of this study (Sub-Parts 1A/1B/1C) are to assess:
1. Incidence of Serious Adverse Events (SAEs), incidence and severity of treatment-emergent AEs (TEAEs) coded to preferred term and system organ class using the Medical Dictionary for Regulatory Activities (MedDRA) and graded according to the CTCAE v5.0, and laboratory abnormalities classified by severity using CTCAE v5.0; and
2. Incidence of TEAEs meeting protocol defined DLT criteria.

The secondary endpoints for the dose escalation phase are to assess:
1. Objective Response Rate (ORR), defined as the proportion of participants with a best overall response of Complete Response (CR) or Partial Response (PR) per RECIST v1.1;
2. PK parameters of SAR444881 (Cₘₐₓ, C_{trough} or Cₜₐᵤ, Tₘₐₓ, T_{1/2}, AUC); and
3. Incidence of anti-drug antibodies (ADA), including neutralizing ADA (nADA), to SAR444881.

The exploratory endpoints for the dose escalation phase are to assess:
1. Correlation/measure of SAR444881 anti-tumor activity by pretreatment level of biomarkers of interest; correlation/measure of association of anti-tumor activity and change from baseline in biomarkers of interest; and
2. Correlation/measure of the association between SAR444881 serum PK, select outcomes, and biomarkers of interest.

### Dose Optimization/Expansion (Part 2)

The primary endpoint for the dose optimization/expansion phase is to assess ORR per RECIST v1.1 (other datapoints may be utilized to supplement assessment of optimal SAR444881 dose).

The secondary endpoints for the dose optimization/expansion phase are to assess:
1. progression-free survival (PFS) defined as the time from the date of first dose of study drug to the date of first documented disease progression or death due to any cause, whichever occurs first, to estimate median PFS using Kaplan-Meier (K-M) methodology; PFS rate at 3, 6, 9, 12 months, and up to 24 months per RECIST v1.1; Disease Control Rate (DCR, rate of CR, PR or Stable Disease (SD)); Duration of Response (DoR) defined as the duration between first documentation of CR or PR to first documentation of disease progression or death from any cause, whichever occurs first;
2. Incidence of SAEs and TEAEs, as described above;
3. PK parameters of SAR444881 (Cₘₐₓ, C_{trough} or Cₜₐᵤ, Tₘₐₓ, T_{1/2}, AUC); and
4. Incidence of ADA, including nADA, to SAR444881.

The exploratory endpoints for the dose optimization/expansion phase are to assess:
1. Correlation/measure of SAR444881 anti-tumor activity by pretreatment level of biomarkers of interest; correlation/measure of association of anti-tumor activity and change from baseline in biomarkers of interest; and
2. Correlation/measure of the association between SAR444881 serum PK, select outcomes, and biomarkers of interest.

### Study Population

Study participants are selected based on the following inclusion and exclusion criteria:

### Inclusion Criteria

1) Type of Participant and Target Disease Characteristics
   a) Patients with unresectable or metastatic disease who are refractory to or are not candidates for standard approved therapy*
   i) Participants with MSI-high (MSI-H) and/or MMR-deficient (dMMR) tumors or with tumor mutational burden-high (TMB-H) [≥10 mutations/megabase (mut/Mb)] tumors should have received pembrolizumab or another anti-PD-1 or anti-PD-L1 therapeutic antibody, if are available as part of the local standard of care, prior to enrollment into this study.
   *Not applicable to Dose Optimization Cohort A1
   b) Participants must have histologic confirmation of malignancy.
   c) Participants must have measurable disease per Response Evaluation Criteria in Solid Tumors (RECIST) v1.1.
   d) All single agent dose escalation (Sub-Part 1A) participants treated at dose levels equal and greater than 1 mg/kg (>1 mg/kg) and all combination dose escalation (Sub-Parts 1B/1C) and dose optimization/expansion phase (Part 2) participants must have at least one lesion accessible for biopsy.
   e) Eastern Cooperative Oncology Group Performance Status (ECOG) of 0 or 1.
   f) Estimated life expectancy of more than 3 months.
   g) Sub-Part 1A (SAR444881 Monotherapy Dose Escalation) Tumor Types:
   i) Breast cancer, cervical cancer, colorectal cancer, adenocarcinoma or squamous cell carcinoma of the esophagus, gastric or gastroesophageal junction adenocarcinoma, squamous cell carcinoma of the head and neck, hepatobiliary cancers (hepatocellular carcinoma, gallbladder cancer, cholangiocarcinoma), non-small cell lung cancer, renal cell carcinoma, squamous cell carcinoma of the skin, or urothelial carcinoma.
   h) Sub-Part 1B (SAR444881 in Combination with Pembrolizumab Dose Escalation) Tumor Types:
   i) Cervical cancer, colorectal cancer, gastric or gastroesophageal junction adenocarcinoma, squamous cell carcinoma of the head and neck, hepatobiliary cancers (hepatocellular carcinoma, gallbladder cancer, cholangiocarcinoma), adenocarcinoma or squamous cell carcinoma of the esophagus, non-small cell lung cancer, pancreatic adenocarcinoma, renal cell carcinoma, squamous cell carcinoma of the skin, triple negative breast cancer, or urothelial carcinoma.
   i) Sub-Part 1C (SAR444881 in Combination with Cetuximab Dose Escalation) Tumor Types:
   i) K-Ras wild-type colorectal cancer (right and left-sided tumors), squamous cell carcinoma of the head and neck, hepatobiliary cancers (hepatocellular carcinoma, gallbladder cancer, cholangiocarcinoma), non-small cell lung cancer, ovarian cancer.
   (1) Participants with BRAF-mutant colorectal cancer are eligible for enrollment.
   ii) Participants with prior exposure to EGFR inhibitors (including cetuximab) are allowed to enroll.
   j) Sub-Part 2A (SAR444881 Dose Optimization) Tumor Types:
   i) Cohort A1:
(1) Histologically or cytologically confirmed diagnosis of Stage IV (per American Joint Committee on Cancer [AJCC] 8th edition) non-squamous NSCLC.
(2) Prior anticancer therapy: Have not received prior systemic therapy for advanced/metastatic NSCLC. Participants who received adjuvant or neoadjuvant therapy are eligible if the adjuvant/neoadjuvant therapy was completed at least 6 months prior to the development of metastatic disease.
ii) Cohort A2:
(1) Histologically or cytologically confirmed diagnosis of Stage IV (per American Joint Committee on Cancer [AJCC] 8th edition) NSCLC.
(2) Prior anticancer therapy: Patients with metastatic NSCLC should have progressed after having received prior benefit from an anti-PD1/PD-L1* containing regimen (SD, partial response [PR], or CR). Patients must have received prior anti-PD1/PD-L1 containing regimen given concurrently or sequentially with a platinum-based chemotherapy. Platinum ineligible patients can enroll after anti-PD1/PD-L1 monotherapy, or anti-PD1/PD-L1 monotherapy followed by a non-platinum form of chemotherapy.
   *If anti-PD1/PD-LI was used beyond initial radiological progression while continuing to use the same anti-PD1/PD-L1 agent used before PD, it is still considered as the same regimen. The site's study team must have reviewed previous tumor assessments (including screening tumor imaging) to determine that radiographic progression has occurred per RECIST 1.1 following initiation of the anti-PD1/PD-1 containing regimen.
iii) Cohort B1:
(1) Histologically or cytologically confirmed diagnosis of GC or Siewert Type 2 & 3 GEJ.
(2) Participants must have MSI status known, determined locally and must have non-MSI-H disease to be eligible.
(3) Participants with unknown HER2/neu status must have their HER2/neu status determined locally. Participants previously treated with a trastuzumab-based regimen must have documentation of disease progression to be eligible.
(4) Prior anticancer therapy: Participants should have received at least two prior lines of treatment, including an anti-PD-1/PD-L1 containing regimen and have progressed after a primary or secondary resistance to an anti-PD-1/PD-L1.
iv) Cohort C1:
(1) Histologically or cytologically confirmed diagnosis of colorectal cancer. Only patients with non-MSI-H disease are eligible.
(2) MSI status: Participants must have MSI status known or determined locally and must have non-MSI-H disease to be eligible.
(3) Prior anticancer therapy: Participants should have failed or relapsed on at least 2 regimens having contained fluoropyrimidine, oxaliplatin, irinotecan, with bevacizumab and/or cetuximab.
k) Sub-Part 2B (SAR444881 Dose Expansion) Tumor Types:
i) Cohort D1:
(1) Patients with only one of the following tumor types:
   Squamous cell carcinoma of the head and neck.
   Gastric or gastroesophageal junction adenocarcinoma.
   Non-small cell lung cancer.
   Cholangiocarcinoma
   (2) Participants must have received and progressed/been intolerant of at least one prior systemic therapy for metastatic and/or unresectable disease.
2) Physical and Laboratory Findings
   a) Participants must have adequate organ function as defined by the following:
   i) Lymphocyte count≥500/mm³
   ii) Neutrophil count≥1,500/mm³
   iii) Platelet count≥100,000/mm³
   iv) Hemoglobin concentration≥9.0 g/dL
   v) AST and ALT≤3.0×ULN or ≤5.0×ULN in patients with hepatocellular carcinoma or if liver metastases are present
   vi) Total bilirubin≤1.5×ULN (except participants with Gilbert Syndrome who must have a total bilirubin level of <3.0×ULN)
   vii) Creatinine clearance (observed or estimated using the Cockcroft-Gault equation)≥45 mL/min
3) Tumor biopsies:
   a) Dose Escalation:
   i) Participants in SAR444881 monotherapy dose escalation 0.1 mg/kg and 0.3 mg/kg dose level cohorts will be asked for their consent for optional pre-and on-treatment biopsies for performance of correlative tissue studies.
   ii) All other participants must consent and will be required to undergo mandatory pre- and on-treatment biopsies for performance of correlative tissue studies.
   iii) All participants will be asked for optional end of treatment biopsy.
   b) Dose Optimization/Expansion:
   i) Mandatory baseline biopsy for participants in all dose optimization/expansion cohorts: Sponsor may discontinue collection once sufficient samples have been collected. Archival tumor tissue samples should be obtained from biopsies done within 6 months, and there should be no systemic anti-cancer therapy between collection of biopsy and enrollment.
   ii) Mandatory on-treatment biopsy for at least 20 participants in all dose optimization/expansion cohorts, if clinically feasible. On-treatment biopsies are otherwise optional per Investigator's discretion and evaluation of all other participants.
   iii) The Sponsor may approve the written request to enroll, on a case-by-case basis, participants with:
   1. location of the tumor not amenable to biopsy due to significant risk, OR
   2. less than required number of slides or archival tumor tissue sample collected more than 6 months prior to enrollment.
4) Age and Reproductive Status
   a) Males and Females, at least 18 years old.
   b) Women of childbearing potential (WOCBP) must have a negative serum or urine pregnancy test (minimum sensitivity 25 IU/L or equivalent units of HCG) within 72 hours prior to the start of study treatment.
   c) Women must not be breastfeeding.
   d) Women of childbearing potential (WOCBP) must agree to follow instructions for method(s) of contraception for the duration of treatment with study treatment and 10 months duration after last dose of study drug.
   e) WOCBP who are continuously not heterosexually active are exempt from contraceptive requirements, but still must undergo pregnancy testing as described in this section.
   f) Males who are sexually active with WOCBP must agree to follow instructions for method(s) of contraception for the duration of treatment with study treatment and 7 months duration after the last dose of study drug. In addition, male participants must be willing to refrain from sperm donation during this time.

### Exclusion Criteria

Exclusionary criteria for participants include the following:
1) Medical Conditions
   a) Active, known or suspected autoimmune disease. Participants with type I diabetes mellitus, hypothyroidism only requiring hormone replacement, skin disorders (such as vitiligo, psoriasis, or alopecia) not requiring systemic treatment, or conditions not expected to recur in the absence of an external trigger are permitted to enroll.
   b) Condition requiring systemic treatment with either corticosteroids (>10 mg daily prednisone equivalent) or other immunosuppressive medications within 14 days of start of study treatment. Inhaled or topical steroids, and adrenal replacement steroid doses <10 mg daily prednisone equivalent, are permitted in the absence of active autoimmune disease.
   c) Brain metastases or leptomeningeal metastases.
   d) Known history of positive test for human immunodeficiency virus (HIV) or known acquired immunodeficiency syndrome (AIDS).
   e) Non-hepatocellular carcinoma participants: acute or chronic hepatitis B virus (HBV) or hepatitis C virus (HCV) infection.
   f) Hepatocellular carcinoma participants: untreated active hepatitis B virus or dual infection with HBV/HCV or other hepatitis combinations or decompensated cirrhosis (Child Pugh B8 and higher).
   g) Prior non-hematological malignancy active within the previous 3 years except for locally curable cancers hat have been apparently cured, such as basal or squamous cell skin cancer, superficial bladder cancer, or carcinoma in situ of the prostate, cervix, or breast.
   h) Prior hematological malignancy active within the previous 5 years.
   i) Participants after solid organ or allogeneic hematopoietic stem cell transplant.
   j) History of life-threatening toxicity related to prior immune therapy (e.g. anti-CTLA-4 or anti-PD-1/PD-L1 treatment or any other antibody or drug specifically targeting co-stimulation of immune cells or immune checkpoint pathways) except those that are unlikely to re-occur with standard countermeasures (e.g. hormone replacement after adrenal crisis).
   k) History of Grade 3 or 4 infusion reaction, Grade 3 or 4 neurological toxicity, Grade 2, 3 or 4 myocarditis, Grade 4 nephritis, AST or ALT increases to more than 8 times ULN or total bilirubin increases to more than 3 times ULN, or Grade 4 colitis related to prior pembrolizumab or other anti-PD(L)-1 antibodies (for Sub-Part 1B).
   l) History of life-threatening toxicity, Grade 3 or 4 infusion reaction, or three or more occurrences of Grade 3 or 4 dermatologic toxicity related to prior cetuximab or other anti-EGFR antibodies (for Sub-Part 1C).
   m) History of clinically significant hypomagnesemia, hypocalcemia, or hypokalemia related to prior cetuximab or other anti-EGFR antibodies (for Sub-Part 1C).
   n) History of Grade 2 or higher pneumonitis or interstitial lung disease.
   o) Unstable or deteriorating cardiovascular disease within the previous 6 months prior to screening including but not limited to the following:
   i) Unstable angina or myocardial infarction;
   ii) Transient ischemic attack (TIA)/cerebrovascular accident (CVA);
   iii) Congestive heart failure (New York Heart Association [NYHA] Class III or IV);
   iv) Uncontrolled clinically significant arrhythmias or arrhythmias requiring medication for rate control.
   p) Any major surgery within 4 weeks of study drug administration. Participants must have recovered from the effects of major surgery at least 14 days before the first dose of the study drug.
   q) Any condition (including medical, emotional, psychiatric, or logistical) that, in the opinion of the Investigator, would preclude the participant from adhering to the protocol or would increase the risk associated with study participation or study drug administration or interfere with the interpretation of study results.
2) Prior/Concomitant Therapy
   a) Immunosuppressive agents or immunosuppressive doses of systemic corticosteroids (except as permitted in the study protocol).
   b) Cytotoxic anti-cancer agents, unless at least 4 weeks have elapsed from last dose of prior anti-cancer therapy and initiation of study therapy.
   c) Non-cytotoxic anti-cancer agents, unless at least 4 weeks or 5 half-lives (whichever is shorter) have elapsed from the last dose of prior anti-cancer therapy and the initiation of study therapy.
   d) Use of other investigational drugs (drugs not marketed for any indication) within 28 days before first study drug administration.
   e) Prior immune therapy treatments (for example, but not limited to: anti-CTLA4, anti-PD-L1, anti-PD-L2, anti-PD-1, IL-2), unless at least 4 weeks or 5 half-lives (whichever is shorter) have elapsed from the last dose of immune therapy and initiation of study therapy.
   f) Prior treatment with macrophage or NK cells activating therapies, e.g. drugs targeting CD-47, SIRPα, KIR, NKG2A.
   g) Inability to comply with restrictions and prohibited treatments.
   h) Treatment with botanical preparations (e.g. herbal supplements or traditional Chinese medicines) intended for general health support or to treat the disease under study within 2 weeks prior to study drug administration.
   i) Administration of a live attenuated vaccine within 28 days prior to the first dose of
   j) Antibiotic use (excluding topical antibiotics)≤14 days prior to first dose of IMP, or any serious systemic fungal, bacterial, viral, or other infection that is not controlled or requires IV or oral antibiotics.
3) Allergies and Adverse Drug Reaction
   a) History of allergy or hypersensitivity to study drug components.
   b) History of any clinically significant drug allergy (such as anaphylaxis, hepatotoxicity, or hypersensitivity reactions) to other monoclonal antibodies.
   4) SAR444881 Dose Optimization (Sub-Part 2A):
   a) Has a known history of Hepatitis B (defined as HBsAg reactive) or known active Hepatitis C virus (defined as HCV RNA [qualitative] is detected) infection. Note: No testing for Hepatitis B and Hepatitis C is required unless mandated by a local health authority.
   b) Cohort A1:
i) Known driver alternations which include epidermal growth factor receptor (EGFR), anaplastic lymphoma kinase (ALK), Proto-oncogene tyrosine-protein kinase (ROS)1, or BRAF mutation for participants with non-squamous NSCLC.
ii) Prior treatment with an agent (approved or investigational) that blocks the PD1/PD-L1 pathway (participants who joined a study with an anti-PD1/PD-L1 but have written confirmation they were on control arm are allowed).
iii) Is unable to interrupt aspirin or other nonsteroidal anti-inflammatory drugs (NSAIDs), other than an aspirin dose≤1.3 g per day, for a 5-day period (8-day period for long-acting agents, such as piroxicam).
   c) Cohorts A1 and A2:
i) Has received radiation therapy to the lung that is >30Gy within 6 months of the first dose of trial treatment.
   d) Cohort C1:
i) Prior treatment with an agent (approved or investigational) that blocks the PD-1/PD-L1 pathway (participants who joined a study with an anti-PD 1/PD-L1 but have written confirmation they were on control arm are allowed).

### Study Interventions Administered

### Dose Escalation Phase (Part 1)

SAR444881 Monotherapy Dose Escalation (Sub-Part 1A): SAR444881 will be administered to participants in escalating doses. Each participant will be administered SAR444881 in two-week intervals at one of the following planned dose levels: 0.1 mg/kg, 0.3 mg/kg, 1 mg/kg, 3 mg/kg, 10 mg/kg, or 20 mg/kg.

SAR444881 Combination with Pembrolizumab Dose Escalation (Sub-Part 1B): SAR444881 will be administered in escalating doses in combination with pembrolizumab. The starting dose of SAR444881 in Sub-Part 1B will be 1 mg/kg or one dose level below the established SAR444881 monotherapy MTD, the lower of the two. The dose of SAR444881 will be escalated up to SAR444881 monotherapy MTD (or MAAD, if no MTD is reached) following the same escalation scheme of Sub-Part 1A. All participants will receive pembrolizumab at a dose of 200 mg. Treatment will be administered in three-week intervals.

SAR444881 Combination with Cetuximab Dose Escalation (Sub-Part 1C): SAR444881 will be administered in escalating doses in combination with cetuximab. The starting dose of SAR444881 in Sub-Part 1C will be 1 mg/kg or one dose level below the established SAR444881 monotherapy MTD, the lower of the two. The dose of SAR444881 will be escalated up to SAR444881 monotherapy MTD (or MAAD, if no MTD is reached) following the same escalation scheme of Sub-Part 1A. All participants will receive cetuximab at a dose of 500 mg/m². Treatment will be administered in two-week intervals. Premedication with a histamine-1 (H1) receptor antagonist will be given prior to each cetuximab dosing.

### Dose Expansion/Optimization Phase (Part 2)

SAR444881 Combination Dose Optimization (Sub-Part 2A): SAR444881 will be administered in combination with one or more of the following: 1) the anti-PDI monoclonal antibody pembrolizumab at a dose of 200 mg; 2) the anti-EGFR monoclonal antibody cetuximab at a dose of 500 mg/m2; and 3) chemotherapy. The chemotherapy administered will include carboplatin at a dose of 10 mg/mL and pemetrexed at a dose of 500 mg/m². The dose level of SAR444881 in Sub-Part 2A will be determined based on data from each sub-part of the dose escalation phase. Sub-Part 2A is divided into four cohorts:
a) Cohort Al: SAR444881 +pembrolizumab+carboplatin+pemetrexed as first line therapy, administered in three-week intervals (disease: non-squamous NSCLC)
b) Cohort A2: SAR444881+pembrolizumab as second or third line therapy, administered in three-week intervals (disease: NSCLC)
c) Cohort B1: SAR444881+pembrolizumab as third line and later therapy, administered in three-week intervals (disease: gastric cancer or gastro-esophageal junction adenocarcinoma)
d) Cohort C1: SAR444881+cetuximab as a third line and later therapy, administered in two-week intervals (colorectal cancer, any RAS status)

SAR444881 Monotherapy Dose Expansion (Sub-Part 2B): SAR444881 will be administered as a monotherapy (Cohort D1). The dose level of SAR444881 in Sub-Part 2B will be determined based on data from the dose escalation phase. SAR444881 will be administered in two-week intervals. The anti-tumor activity of SAR444881 monotherapy will be evaluated in one of the following indications: advanced stage squamous cell carcinoma of the head and neck, gastric or gastroesophageal junction adenocarcinoma, cholangiocarcinoma, and non-squamous non-small cell lung cancer.

Study treatments and modes of administration are summarized in Table 1 below:

TABLE 1
Study Treatments

| | | | Route of |
|---|---|---|---|
| | | Frequency of | Admini- |
| Study Drug | Dosage Level(s) | Administration | stration |
| SAR444881 | Escalation: | Sub-Parts | IV |
| | Sub-Part 1A: 0.1 mg/kg, | 1A/1C and | |
| | 0.3 mg/kg, | Part 2A/B | |
| | 1 mg/kg, 3 mg/kg, | Cohorts C1 & | |
| | 10 mg/kg, and 20 mg/kg | D1: Q2W | |
| | Sub-Parts 1B/1C: | Sub-Part 1B | |
| | 1 mg/kg, 3 mg/kg, | and Sub- | |
| | 10 mg/kg, and 20 mg/kg | Part 2A | |
| | Optimization/Expansion: | Cohorts | |
| | Up to two | A1, A2, | |
| | recommended doses; | & B1: Q3W | |
| | neither to exceed | | |
| | MTD/MAAD | | |
| Pembrolizumab | 200 mg | Q3W | IV |
| Cetuximab | Escalation and | Q2W | IV |
| | Optimization Cohort | | |
| | C1: 500 mg/m² | | |
| Carboplatin | AUC of 5 | Q3W for | IV |
| | | 4 cycles | |
| Pemetrexed | 500 mg/m² | Q3W | IV |

SAR444881 will be infused over 60 minutes at all dosage levels, except for the 0.1 mg/kg dosage, which will be infused over 30 minutes. Pembrolizumab, cetuximab, carboplatin, and pemetrexed will be administered according to their recommended dosage and administration including use of premedication. No dose reduction for pembrolizumab is allowed. Cetuximab dosage may be modified for adverse reactions management in accordance with the drug's Prescribing Information.

For combination cohorts, pembrolizumab or cetuximab will be infused first, followed by SAR444881. The SAR444881 infusion will start at least 30 minutes after completion of the pembrolizumab infusion and at least 60 minutes after the cetuximab infusion.

All participants administered cetuximab will be premedicated with a histamine-1 (H₁) receptor antagonist intravenously (e.g., diphenhydramine 50 mg) 30-60 minutes prior to each cetuximab dosing. Premedication for patients in cohort A1 receiving pemetrexed include folic acid, vitamin B12, and dexamethasone.

For all study participants, SAR444881 infusions will require a 60-minute observation period following the completion of the infusion for the first 2 doses and a 30-minute observation period for all subsequent doses.

Cohort A1 dosing sequence: pembrolizumab, pemetrexed premedication, pemetrexed, carboplatin, and SAR444881 (start of SAR444881 infusion should be at least 30minutes after completion of pembrolizumab infusion).

### Example 2: Study of SAR444881 Administered Alone and in Combination with Other Therapeutics in Participants with Advanced Solid Tumors

This Example outlines the protocol and preliminary results for an open-label, multicenter, Phase 1/2, first-in-human study of the safety, tolerability, and anti-tumor activity of intravenous SAR444881, a humanized monoclonal IgG₄ antibody targeting ILT2. The study will evaluate SAR444881, as a single agent and when combined with pembrolizumab or with cetuximab, for treatment of cancer patients with advanced solid tumors where no other standard of care treatment option is available. Given the known mechanism of action of ILT2 and its potential role in cancer, the study focuses on tumor types known to express HLA-G. Tumor types included in SAR444881-cetuximab combination cohorts are tumors known to over-express the EGFR protein.

### Study Design

The proposed study design is as described in Example 1.

### Objectives

The primary, secondary, and exploratory objectives of the dose escalation phase of this study (Part 1) are as described in Example 1.

The primary objectives of the dose expansion/optimization phase of this study (Part 2) are to assess the preliminary antitumor activity of SAR444881 (alone or in combination with pembrolizumab, cetuximab, or pembrolizumab and chemotherapy), and to define the optimal dose of SAR444881 in combination with pembrolizumab, cetuximab, or pembrolizumab and chemotherapy.

The secondary objectives of the dose expansion/optimization phase of this study are:
To assess additional parameters of anti-tumor activity of SAR444881, including median progression free survival (PFS), PFS rate at defined intervals (3, 6, 9 and 12 months, and up to 24 months, per RECIST v1.1), and duration of response (DoR);
To further assess the safety and tolerability of SAR444881;
To further characterize the PK of SAR444881; and
To further characterize the immunogenicity of SAR444881.

The exploratory objectives of the dose expansion/optimization phase of this study are:
To explore the potential associations between SAR444881 anti-tumor activity and select biomarker measures in the tumor and peripheral blood;
To explore the associations between SAR444881 serum PK, safety, efficacy, and clinical biomarkers; and.
To explore the effect of the study treatment on participant-reported outcomes (PROs).

### Endpoints

### Dose Escalation (Part 1)

The primary endpoints for the dose escalation phase of this study (Sub-Parts 1A/1B/1C) are to assess:
3. Incidence of Serious Adverse Events (SAEs), incidence and severity of treatment-emergent AEs (TEAEs) coded to preferred term and system organ class using the Medical Dictionary for Regulatory Activities (MedDRA) and graded according to the CTCAE v5.0, and laboratory abnormalities classified by severity using CTCAE v5.0; and
4. Incidence of TEAEs meeting protocol defined DLT criteria.

The secondary endpoints for the dose escalation phase are to assess:
4. Objective Response Rate (ORR), defined as the proportion of participants with a best overall response of Complete Response (CR) or Partial Response (PR) per RECIST v1.1;
5. PK parameters of SAR444881 (Cₘₐₓ, C_{trough}, Tₘₐₓ, T_{1/2}, AUC); and
6. Incidence of anti-drug antibodies (ADA) to SAR444881.

The exploratory endpoints for the dose escalation phase are to assess:
3. Correlation/measure of SAR444881 anti-tumor activity by pretreatment level of biomarkers of interest; correlation/measure of association of anti-tumor activity and change from baseline in biomarkers of interest; and
4. Correlation/measure of the association between SAR444881 serum PK, select outcomes, and biomarkers of interest.

### Dose Optimization/Expansion (Part 2)

The primary endpoint for the dose optimization/expansion phase is to assess ORR per RECIST v1.1 (other datapoints may be utilized to supplement assessment of optimal SAR444881 dose).

The secondary endpoints for the dose optimization/expansion phase are to assess:
5. progression-free survival (PFS) defined as the time from the date of first dose of study drug to the date of first documented disease progression or death due to any cause, whichever occurs first, to estimate median PFS using Kaplan-Meier (K-M) methodology; PFS rate at 3, 6, 9, 12 months, and up to 24 months per RECIST v1.1; Disease Control Rate (DCR, rate of CR, PR or Stable Disease (SD)); Duration of Response (DoR) defined as the duration between first documentation of CR or PR to first documentation of disease progression or death from any cause, whichever occurs first;
6. Incidence of SAEs and TEAEs, as described above;
7. PK parameters of SAR444881 (Cₘₐₓ, C_{trough}, Tₘₐₓ, T_{1/2}, AUC); and
8. Incidence of ADA to SAR444881.

The exploratory endpoints for the dose optimization/expansion phase are to assess:
3. Correlation/measure of SAR444881 anti-tumor activity by pretreatment level of biomarkers of interest; correlation/measure of association of anti-tumor activity and change from baseline in biomarkers of interest; and
4. Correlation/measure of the association between SAR444881 serum PK, select outcomes, and biomarkers of interest.

### Study Population

Study participants are selected based on the following inclusion and exclusion criteria:

### Inclusion Criteria

1) Type of Participant and Target Disease Characteristics
a) Participants with unresectable or metastatic disease who are refractory to or are not candidates for standard approved therapy*
i) Participants with MSI-high (MSI-H) and/or MMR-deficient (dMMR) tumors or with tumor mutational burden-high (TMB-H) [>10 mutations/megabase (mut/Mb)] tumors should have received pembrolizumab or another anti-PD-1 or anti-PD-L1 therapeutic antibody, if are available as part of the local standard of care, prior to enrollment into this study.
   *all sub-parts and cohorts aside from Dose Optimization Cohort A1; Cohort A1 participants remain eligible to receive standard approved therapy
b) Participants must have histologic confirmation of malignancy.
c) Participants must have measurable disease per Response Evaluation Criteria in Solid Tumors (RECIST) v1.1.
d) All single agent dose escalation (Sub-Part 1A) participants treated at dose levels equal and greater than 1 mg/kg (>1 mg/kg) and all combination dose escalation (Sub-Parts 1B/1C) and dose optimization/expansion phase (Part 2) participants must have at least one lesion accessible for biopsy.
e) Eastern Cooperative Oncology Group Performance Status (ECOG) of 0 or 1.
f) Estimated life expectancy of more than 3 months.
g) Sub-Part 1A-1C tumor types are as described in Example 1.
h) Sub-Part 2A (SAR444881 Dose Optimization) Tumor Types:
i) Cohort A1:
(1) Histologically or cytologically confirmed diagnosis of Stage IV (per American Joint Committee on Cancer [AJCC] 8th edition) non-squamous NSCLC.
(2) Prior anticancer therapy: Have not received prior systemic therapy for advanced/metastatic NSCLC. Participants who received adjuvant or neoadjuvant therapy are eligible if the adjuvant/neoadjuvant therapy was completed at least 6 months prior to the development of metastatic disease.
ii) Cohort B1:
(1) Histologically or cytologically confirmed diagnosis of GC or Siewert Type 2 & 3 GEJ.
(2) Participants must have MSI status known, determined locally and must have non-MSI-H disease to be eligible.
(3) Participants with unknown HER2/neu status must have their HER2/neu status determined locally. Participants previously treated with a trastuzumab-based regimen must have documentation of disease progression to be eligible.
(4) Prior anticancer therapy: Participants should have received at least one prior line of treatment, including an anti-PD-1/PD-L1 containing regimen and have progressed after a primary or secondary resistance to an anti-PD-1/PD-L1.
iii) Cohort C1:
(1) Histologically or cytologically confirmed diagnosis of colorectal cancer. Only participants with non-MSI-H disease are eligible.
(2) MSI status: Participants must have MSI status known or determined locally and must have non-MSI-H disease to be eligible.
(3) Participants with RAS-mutant and BRAF-mutant colorectal cancer are eligible for enrollment.
(4) Prior anticancer therapy: Participants should have failed or relapsed on at least one prior regimen.
   a) Participants with RAS wild type colorectal cancer should have either failed or relapsed on or be ineligible for treatment with 5-fluorouracil, irinotecan, oxaliplatin, bevacizumab and anti-EGFR.
   b) Participants with RAS-mutant colorectal cancer are eligible for enrollment; participants with RAS-mutant disease should have either failed or relapsed on or be ineligible for bevacizumab treatment.
   c) Participants with BRAF-mutant colorectal cancer are eligible for enrollment; participants should have either failed or relapsed on treatment or be ineligible for treatment with encorafenib or other BRAF targeted therapies.
   d) Once 10 participants that previously have not been exposed to cetuximab or other anti-EGFR therapy have been enrolled, enrollment will be limited to participants who have received cetuximab or other anti-EGFR therapy as part of their last line of treatment.
iv) Cohort E1
(1) Histologically or cytologically confirmed NSCLC, Stage IV (per American Joint Committee on Cancer [AJCC] 8th edition). Up to 10 participants with non-squamous NSCLC will be enrolled in Part 2, Stage 1, with the remaining enrollment allocated to participants with squamous NSCLC.
(2) Prior anticancer therapy: Participants should have failed or relapsed on at least one prior regimen.
(3) Participants with known driver alterations which include epidermal growth factor receptor (EGFR), anaplastic lymphoma kinase (ALK), Proto-oncogene tyrosine-protein kinase (ROS), BRAF, or RET mutation are eligible for inclusion. Participants with driver mutations must have failed or relapsed on or are ineligible for available targeted therapies.
i) Sub-Part 2B (SAR444881 Dose Expansion) Tumor Types:
i) Cohort D1:
(1) Histologically or cytologically confirmed advanced cholangiocarcinoma.
(2) Participants must have received and progressed/been intolerant of at least one prior systemic therapy for metastatic and/or unresectable disease for whom all standard of care therapies have failed.
2) Physical and Laboratory Findings
a) Participants must have adequate organ function as defined by the following:
i) Lymphocyte count≥500/mm³
ii) Neutrophil count≥1,500/mm³
iii) Platelet count≥100,000/mm³
iv) Hemoglobin concentration≥9.0g/dL
   a. Criteria must be met without packed red blood cell transfusion within the prior 2 weeks. Participants can be on stable doses of erythropoietin (>approximately 3 months).
v) AST and ALT≤2.5×ULN or ≤5.0×ULN in participants with hepatocellular carcinoma, advanced cholangiocarcinoma, or if liver metastases are present
vi) Total bilirubin≤1.5×ULN (except participants with Gilbert Syndrome who must have a total bilirubin level of <3.0×ULN)
vii) Creatinine clearance (observed or estimated using the Cockcroft-Gault equation)≥45 mL/min (Cohort A1 and D1 only)
3) Tumor biopsies:
a) Dose Escalation:
i) Participants in SAR444881 monotherapy dose escalation 0.1 mg/kg and 0.3 mg/kg dose level cohorts will be asked for their consent for optional pre-and on-treatment biopsies for performance of correlative tissue studies.
ii) All other participants must consent and will be required to undergo mandatory pre- and on-treatment biopsies for performance of correlative tissue studies.
iii) All participants will be asked for optional end of treatment biopsy.
b) Dose Optimization/Expansion:
i) Mandatory baseline biopsy for participants with low risk biopsies (i.e., expected rate of major complications <0.5%, e.g., blood, bone marrow, skin, and superficial masses) in all dose optimization/expansion cohorts: Tissue must be available for submission to the Sponsor. If sufficient sample is not available from biopsies collected in the last 6 months with no intervening therapy prior to consent, then the participant must be willing and able to undergo a biopsy for this collection.
ii) Moderate risk biopsies (i.e., expected rate of major complications 0.5-1.5%, e.g., intra-abdominal, some intrathoracic, and low risk procedures in participants with increased risk of bleeding or infection) are optional, and not mandatory. High risk biopsies (i.e., expected rate of major complications >1.5%) are not acceptable in this trial based on the current trial objectives.
iii) On-treatment biopsy for at least 20 participants in all dose optimization/expansion cohorts, if clinically feasible, but may be optional before 20 biopsies have been obtained based on the totality of data at the sole discretion of the Sponsor.
iv) If imaging is required to obtain biopsy material, the biopsy should be performed using ultrasound guidance if feasible to prevent additional study-related radiation dose.
4) Age and Reproductive Status
   a) Males and Females, at least 18 years old.
   b) Women of childbearing potential (WOCBP) must have a negative serum or urine pregnancy test (minimum sensitivity 25 IU/L or equivalent units of HCG) within 72 hours prior to the start of study treatment.
   c) Women must not be breastfeeding.
   d) Women of childbearing potential (WOCBP) must agree to follow instructions for method(s) of contraception for the duration of treatment with study treatment and 10 months duration after last dose of study drug.
   e) WOCBP who are continuously not heterosexually active are exempt from contraceptive requirements, but still must undergo pregnancy testing as described in this section.
   f) Males who are sexually active with WOCBP must agree to follow instructions for method(s) of contraception for the duration of treatment with study treatment and 7 months duration after the last dose of study drug. In addition, male participants must be willing to refrain from sperm donation during this time.

### Exclusion Criteria

Exclusionary criteria for participants include the following:
1) Medical Conditions
a) Active, known or suspected autoimmune disease. Participants with type I diabetes mellitus, hypothyroidism only requiring hormone replacement, skin disorders (such as vitiligo, psoriasis, or alopecia) not requiring systemic treatment, or conditions not expected to recur in the absence of an external trigger are permitted to enroll.
b) Condition requiring systemic treatment with either corticosteroids (>10 mg daily prednisone equivalent) or other immunosuppressive medications within 14 days of start of study treatment. Inhaled or topical steroids, and adrenal replacement steroid doses <10 mg daily prednisone equivalent, are permitted in the absence of active autoimmune disease.
c) Has known active CNS metastases and/or carcinomatous meningitis. Participants with previously treated brain metastases may participate provided they are radiologically stable, (i.e., without evidence of progression) for at least four weeks by repeat imaging (note: the repeat imaging should be performed during study screening), clinically stable, and without requirement of steroid treatment for at least 14 days before the first dose of study intervention.
d) Known history of positive test for human immunodeficiency virus (HIV) or known acquired immunodeficiency syndrome (AIDS).
e) Non-hepatocellular carcinoma participants: acute or chronic hepatitis B virus (HBV) or hepatitis C virus (HCV) infection.
f) Hepatocellular carcinoma participants: untreated active hepatitis B virus or dual infection with HBV/HCV or other hepatitis combinations or decompensated cirrhosis (Child Pugh B8 and higher).
g) Prior non-hematological malignancy active within the previous 3 years except for locally curable cancers that have been apparently cured, such as basal or squamous cell skin cancer, superficial bladder cancer, or carcinoma in situ of the prostate, cervix, or breast.
h) Prior hematological malignancy active within the previous 5 years.
i) Participants after solid organ or allogeneic hematopoietic stem cell transplant.
j) History of life-threatening toxicity related to prior immune therapy (e.g. anti-CTLA-4 or anti-PD-1/PD-L1 treatment or any other antibody or drug specifically targeting co-stimulation of immune cells or immune checkpoint pathways) except those that are unlikely to re-occur with standard countermeasures (e.g. hormone replacement after adrenal crisis).
k) History of Grade 3 or 4 infusion reaction, Grade 3 or 4 neurological toxicity, Grade 2, 3 or 4 myocarditis, Grade 4 nephritis, AST or ALT increases to more than 8 times ULN or total bilirubin increases to more than 3 times ULN, or Grade 4 colitis related to prior pembrolizumab or other anti-PD (L)-1 antibodies (for Sub-Part 1B, and Part 2 Cohorts A1 and B1).
1) History of life-threatening toxicity, Grade 3 or 4 infusion reaction, or three or more occurrences of Grade 3 or 4 dermatologic toxicity related to prior cetuximab or other anti-EGFR antibodies (for Sub-Part 1C, and Part 2 Cohorts C1 and E1).
m) History of clinically significant hypomagnesemia, hypocalcemia, or hypokalemia related to prior cetuximab or other anti-EGFR antibodies (for Sub-Part 1C, and Part 2 Cohorts C1 and E1).
n) History of Grade 2 or higher pneumonitis or interstitial lung disease.
0) Unstable or deteriorating cardiovascular disease within the previous 6 months prior to screening including but not limited to the following:
i) Unstable angina or myocardial infarction;
ii) Transient ischemic attack (TIA)/cerebrovascular accident (CVA);
iii) Congestive heart failure (New York Heart Association [NYHA] Class III or IV);
iv) Uncontrolled clinically significant arrhythmias or arrhythmias requiring medication for rate control.
p) Any major surgery within 4 weeks of study drug administration. Participants must have recovered from the effects of major surgery at least 14 days before the first dose of the study drug.
q) Any condition (including medical, emotional, psychiatric, or logistical) that, in the opinion of the Investigator, would preclude the participant from adhering to the protocol or would increase the risk associated with study participation or study drug administration or interfere with the interpretation of study results.
2) Prior/Concomitant Therapy
a) Immunosuppressive agents or immunosuppressive doses of systemic corticosteroids (except as permitted in the study protocol).
b) Cytotoxic anti-cancer agents, unless at least 4 weeks have elapsed from last dose of prior anti-cancer therapy and initiation of study therapy.
c) Non-cytotoxic anti-cancer agents, unless at least 4 weeks or 5 half-lives (whichever is shorter) have elapsed from the last dose of prior anti-cancer therapy and the initiation of
d) Use of other investigational drugs (drugs not marketed for any indication) within 28 days before first study drug administration.
e) Prior immune therapy treatments (for example, but not limited to: anti-CTLA4, anti-PD-L1, anti-PD-L2, anti-PD-1, IL-2), unless at least 4 weeks or 5 half-lives (whichever is shorter) have elapsed from the last dose of immune therapy and initiation of study therapy.
f) Prior treatment with macrophage or NK cells activating therapies, e.g. drugs targeting CD-47, SIRPα, KIR, NKG2A.
g) Inability to comply with restrictions and prohibited treatments.
h) Treatment with botanical preparations (e.g. herbal supplements or traditional Chinese medicines) intended for general health support or to treat the disease under study within 2 weeks prior to study drug administration.
i) Administration of a live attenuated vaccine within 28 days prior to the first dose of study therapy.
j) Antibiotic use (excluding topical antibiotics)≤14 days prior to first dose of IMP, or any serious systemic fungal, bacterial, viral, or other infection that is not controlled or requires IV or oral antibiotics.
k) Has received prior radiotherapy within two weeks of start of study treatment or has had a history of radiation pneumonitis.
   Note: Participants must have recovered from all radiation-related toxicities and not require corticosteroids. A one-week washout is permitted for palliative radiation (≤2 weeks of radiotherapy) to non-CNS disease.
3) Allergies and Adverse Drug Reaction
a) History of allergy or hypersensitivity to study drug components.
b) Has severe hypersensitivity (>Grade 3) to pembrolizumab and/or any of its excipients.
c) History of any clinically significant drug allergy (such as anaphylaxis, hepatotoxicity, or hypersensitivity reactions) to other monoclonal antibodies.
4) Pregnancy Exclusion
a) A WOCBP who has a positive urine pregnancy test (within 72 hours) prior to treatment. If the urine test is positive or cannot be confirmed as negative, a serum pregnancy test will be required.
5) SAR444881 Dose Optimization (Sub-Part 2A):
a) Has a known history of Hepatitis B (defined as HBsAg reactive) or known active Hepatitis C virus (defined as HCV RNA [qualitative] is detected) infection. Note: No testing for Hepatitis B and Hepatitis C is required unless mandated by a local health authority.
b) Cohort A1:
i) Known driver alterations which include epidermal growth factor receptor (EGFR), anaplastic lymphoma kinase (ALK), Proto-oncogene tyrosine-protein kinase (ROS)1, BRAF, or RET mutation for participants with non-squamous NSCLC.
ii) Prior treatment with an agent (approved or investigational) that blocks the PD1/PD-L1 pathway (participants who joined a study with an anti-PD1/PD-L1 but have written confirmation they were on control arm are allowed).
iii) Is unable to interrupt aspirin or other nonsteroidal anti-inflammatory drugs (NSAIDs), other than an aspirin dose ≤1.3 g per day, for a 5-day period (8-day period for long-acting agents, such as piroxicam).
c) Cohorts A1 and E1:
i) Has received radiation therapy to the lung that is >30Gy within 6 months of the first dose of trial treatment.
d) Cohort C1:
i) Prior treatment with an agent (approved or investigational) that blocks the PD-1/PD-L1 pathway (participants who joined a study with an anti-PD 1/PD-L1 but have written confirmation they were on control arm are allowed).
ii) The participant has clinically relevant coronary artery disease or history of myocardial infarction in the last 12 months or high risk of uncontrolled arrhythmia or uncontrolled cardiac insufficiency.
iii) The participant has uncontrolled or poorly controlled hypertension (>180 mmHg systolic or >130 mmHg diastolic).
iv) The participant has a history of allergic reactions attributed to compounds of chemical or biologic composition similar to those of cetuximab, or if the participant had red meat allergy/tick bite history.

### Study Interventions Administered

### Dose Escalation Phase (Part 1)

The study interventions administered are as described for the Dose Escalation Phase (Part 1) in Example 1.

### Dose Expansion/Optimization Phase (Part 2)

A two-stage design will be implemented in Part 2 to conduct dose optimization for each indication with combination therapy:

Stage 1 (Preliminary Assessment): Enroll 20 participants and treat the participants at one potential recommended dose identified from dose escalation in each indication to get preliminary efficacy/safety, PK, and PD data.
Stage 2 (Randomization): Randomize 40 participants 1:1 into the two recommended doses with each indication that showed activity in Stage 1 to support selection of optimal dose for the indication. In addition, it is possible that emerging data from Stage 1 may indicate the need to deviate from this randomization scenario. Based on the totality of data across the study as a whole and with agreement of the SRC, the Sponsor will retain flexibility on the design of the Stage 2, which may include, among other possibilities, a single dose level rather than randomization, or termination of the cohort prior to entry to Stage 2.

SAR444881 Combination Dose Optimization (Sub-Part 2A): SAR444881 will be administered in combination with one or more of the following: 1) the anti-PDI monoclonal antibody pembrolizumab at a dose of 200 mg; 2) the anti-EGFR monoclonal antibody cetuximab at a dose of 500 mg/m²; and 3) chemotherapy. The chemotherapy administered will include carboplatin at a dose of 10 mg/mL and pemetrexed at a dose of 500 mg/m². The dose level of SAR444881 in Sub-Part 2A will be determined based on data from each sub-part of the dose escalation phase.

Sub-Part 2A is divided into four cohorts:
a) Cohort A1: SAR444881+pembrolizumab+carboplatin+pemetrexed as first line therapy, administered in three-week intervals (disease: non-squamous NSCLC)
b) Cohort B1: SAR444881+pembrolizumab as second line and later therapy, administered in three-week intervals (disease: gastric cancer or gastro-esophageal junction adenocarcinoma)
c) Cohort C1: SAR444881+cetuximab as a second line and later therapy, administered in two-week intervals (disease: colorectal cancer, any RAS status)
d) Cohort E1: SAR444881+cetuximab as a second line and later therapy, administered in two-week intervals (disease: NSCLC)

The study includes a safety run-in and core phase for Cohort A1 (SAR444881+pembrolizumab+carboplatin+pemetrexed for non-squamous NSCLC). The initial SAR444881 dose tested in the safety run-in for Cohort A1 will be 10 mg/kg. If unacceptable toxicity is observed at the 10 mg/kg dose level, then the safety run-in dose will be de-escalated to 3 mg/kg SAR444881.

SAR444881 Monotherapy Dose Expansion (Sub-Part 2B): SAR444881 will be administered as a monotherapy (Cohort D1), as a second line and later therapy. The dose level of SAR444881 in Sub-Part 2B will be determined based on data from the dose escalation phase. SAR444881 will be administered in two-week intervals. The anti-tumor activity of SAR444881 monotherapy will be evaluated in cholangiocarcinoma. Study treatments and modes of administration are summarized in Table 2 below:

TABLE 2
Study Treatments

| | | | Route of |
|---|---|---|---|
| | | Frequency of | Admini- |
| Study Drug | Dosage Level(s) | Administration | stration |
| SAR444881 | Escalation: | Sub-Parts 1A/1C | IV |
| | Sub-Part 1A: 0.1 mg/kg, | and Part 2 | |
| | 0.3 mg/kg, 1 mg/kg, | Cohorts C1, D1, | |
| | 3 mg/kg, 10 mg/kg, and | and E1: Q2W | |
| | 20 mg/kg | Sub-Part 1B | |
| | Sub-Parts 1B/1C: | and Part 2 | |
| | 1 mg/kg, 3 mg/kg, | Cohorts A1 | |
| | 10 mg/kg, and 20 mg/kg | and B1: | |
| | Optimization/Expansion: | Q3W | |
| | (Part 2, Stage 1): | | |
| | 10 mg/kg | | |
| | (all cohorts) | | |
| Pembrolizumab | 200 mg | Q3W | IV |
| Cetuximab | Escalation and | Q2W | IV |
| | Optimization | | |
| | Cohort C1: 500 mg/m² | | |
| Carboplatin | AUC of 5 | Q3W for 4 cycles | IV |
| Pemetrexed | 500 mg/m² | Q3W | IV |

SAR444881 will be infused over 60 minutes at all dosage levels, except for the 0.1 mg/kg dosage, which will be infused over 30 minutes. Pembrolizumab, cetuximab, carboplatin, and pemetrexed will be administered according to their recommended dosage and administration including use of premedication. No dose reduction for pembrolizumab is allowed. Cetuximab dosage may be modified for adverse reactions management in accordance with the drug's Prescribing Information.

For combination cohorts, pembrolizumab or cetuximab will be infused first, followed by SAR444881. The SAR444881 infusion will start at least 30 minutes after completion of the pembrolizumab infusion and at least 60 minutes after the cetuximab infusion.

All participants administered cetuximab will be premedicated with a histamine-1 (H₁) receptor antagonist intravenously (e.g., diphenhydramine 50 mg) 30-60 minutes prior to each cetuximab dosing. Premedication for participants in cohort A1 receiving pemetrexed include folic acid, vitamin B12, and dexamethasone.

For all study participants, SAR444881 infusions will require a 60-minute observation period following the completion of the infusion for the first 2 doses and a 30-minute observation period for all subsequent doses.

Cohort A1 dosing sequence: pembrolizumab, pemetrexed premedication, pemetrexed, carboplatin, and SAR444881 (start of SAR444881 infusion should be at least 30minutes after completion of pembrolizumab infusion).

### Example 3: Safety and Efficacy Data for SAR444881 Monotherapy Treatment (Sub-Part 1A)

This Example describes preliminary safety, pharmacokinetic, and efficacy data for the first five dose level cohorts (0.1, 0.3, 1, 3, and 10 mg/kg) of Part 1-Sub-Part 1A of the clinical study as described in Example 1.

The primary objectives of the study were to assess the safety and tolerability of SAR444881 when administered alone, and to determine the maximum tolerated dose (MTD) or maximum administered dose (MAAD) and the recommended Phase 2 dose (RP2D) of SAR444881 when administered alone. The secondary objectives of the study were to assess the preliminary anti-tumor activity of SAR444881 and to characterize the PK and immunogenicity of SAR444881. The exploratory objectives were to explore potential associations between SAR444881 anti-tumor activity and select biomarker measures in the tumor and peripheral blood, and to explore the associations between SAR444881 serum PK, safety, efficacy, and clinical biomarkers.

In Sub-Part 1A, each patient was assigned to receive a single dose level of SAR444881 alone, with dose escalation between cohorts run with a standard 3+3 design for the five tested doses (0.1 mg/kg, 0.3 mg/kg, 1 mg/kg, 3 mg/kg and 10 mg/kg). A sentinel patient was recruited at each dose level and followed for toxicities for 72 hours before two additional patients were enrolled to the same cohort, for a total of at least three patients per dose level. Enrollment into the next cohort did not begin until the completion of a 28-day observation period for the last patient of the previous cohort. At any dose level, if 1 patient has a dose-limiting toxicity (DLT), a total of 6 patients were to be treated with that dose. If only 1 of the 6 subjects had a DLT, then the next cohort was to be treated at the next dose level. If 2 or more DLTs occurred within a cohort, then that dose level was be considered above the MTD (the highest dose where no more than 1 of 6 subjects has experienced a DLT), and additional subjects were to be enrolled at the previous lower (tolerated) dose level until that cohort had 6 subjects. This lower dose level was considered the MTD if ≤1 of the 6 subjects had a DLT.

This study enrolled 20 advanced cancer patients with unresectable or metastatic disease who are refractory to or are not candidates for standard approved therapy. Mean patient age was 59.4+13.9 years and the majority of patients were male (75%) and White (95%) and non-or former smokers (90%). The most common concomitant morbidity was hypertension, which affected 8/20 patients. Most (80%) patients presented with metastatic tumors, with colorectal cancer and non-small cell lung cancer being the most common cancer types (35% and 20%, respectively), while the remaining had unresectable tumors. All patients had undergone at least two lines of prior treatment; the median number of prior treatment lines was 3.5. Median time from cancer diagnosis for the entire patient population was 42.4 months and was lower in the 0.01 mg/kg and 1 mg/kg treatment groups (28.7 months and 12.7 months, respectively) as compared to the other cohorts. See, e.g., Table 3.

The mean number of doses administered in all cohorts ranged between 3.6 (0.1mg/kg) and 8.5 (3 mg/kg), with one patient in the 3 mg/kg cohort receiving 18 doses of the study drug as of the cutoff date. Accordingly, mean duration of treatment to date ranged between 7.3 and 17.5 weeks, with the minimum duration being 2 weeks and the maximum duration being 39 weeks.

TABLE 3
Demographic and Baseline Characteristics of Treatment Cohorts

| | Treatment Group | | | | | | | | | | | |
|---|---|---|---|---|---|---|---|---|---|---|---|---|
| | 0.1 mg/kg | | 0.3 mg/kg | | 1 mg/kg | | 3 mg/kg | | 10 mg/kg | | Total | |
| | (N = 5) | | (N = 3) | | (N = 3) | | (N = 6) | | (N = 3) | | (N = 20) | |
| Age (years) | | | | | | | | | | | | |
| Mean (StD) | 58.4 | (12.3) | 72.3 | (13.0) | 54.3 | (19.6) | 54.7 | (13.4) | 62.7 | (12.1) | 59.4 | (13.9) |
| Median | 59.0 | | 73.0 | | 56.0 | | 59.5 | | 67.0 | | 60.0 | |
| Minimum, Maximum | 39.0, 72.0 | | 59.0, 85.0 | | 34.0, 73.0 | | 28.0, 63.0 | | 49.0, 72.0 | | 28.0, 85.0 | |
| Sex, n (%) | | | | | | | | | | | | |
| Male | 3 | (60.0) | 3 | (100) | 2 | (66.7) | 5 | (83.3) | 2 | (66.7) | 15 | (75.0) |
| Female | 2 | (40.0) | — | | 1 | (33.3) | 1 | (16.7) | 1 | (33.3) | 5 | (25.0) |
| Race, n (%) | | | | | | | | | | | | |
| Black/African American | 1 | (20.0) | — | | — | | — | | — | | 1 | (5.0) |
| White | 4 | (80.0) | 3 | (100) | 3 | (100) | 6 | (100) | 3 | (100) | 19 | (95.0) |
| Medical History, n (%)* | | | | | | | | | | | | |
| Hypothyroidism | 1 | (20.0) | — | | — | | 2 | (33.3) | 2 | (66.7) | 5 | (25.0) |
| Abdominal pain | 1 | (20.0) | 2 | (66.7) | 1 | (33.3) | 1 | (16.7) | — | | 5 | (25.0) |
| Constipation | 3 | (60.0) | 1 | (33.3) | 2 | (66.7) | — | | — | | 6 | (30.0) |
| Diabetes mellitus | 1 | (20.0) | 2 | (66.7) | — | | 1 | (16.7) | 1 | (33.3) | 5 | (25.0) |
| Hyperlipidemia | — | | 2 | (66.7) | 1 | (33.3) | 1 | (16.7) | 1 | (33.3) | 5 | (25.0) |
| Neuropathy peripheral | 2 | (40.0) | 1 | (33.3) | — | | 2 | (33.3) | — | | 5 | (25.0) |
| Insomnia | — | | 3 | (100) | 1 | (33.3) | 1 | (16.7) | — | | 5 | (25.0) |
| Hypertension | 3 | (100) | 1 | (33.3) | — | | 3 | (50.0) | 1 | (33.3) | 8 | (40.0) |
| Cancer Status, n (%) | | | | | | | | | | | | |
| Metastatic | 3 | (60.0) | 3 | (100) | 2 | (66.7) | 5 | (83.3) | 3 | (100) | 16 | (80.0) |
| Unresectable | 2 | (40.0) | — | | 1 | (33.3) | 1 | (16.7) | — | | 4 | (20.0) |
| Cancer Type, n (%) | | | | | | | | | | | | |
| Cervical cancer | 1 | (20.0) | — | | — | | — | | — | | 1 | (5.0) |
| Cholangiocarcinoma | — | | — | | 1 | (33.3) | 2 | (33.3) | — | | 3 | (15.0) |
| Colorectal cancer | 1 | (20.0) | 2 | (66.7) | 1 | (33.3) | 2 | (33.3) | 1 | (33.3) | 7 | (35.0) |
| Gallbladder cancer | 1 | (20.0) | — | | — | | — | | — | | 1 | (5.0) |
| Gastric adenocarcinoma | — | | 1 | (33.3) | — | | — | | — | | 1 | (5.0) |
| Gastroesophageal junction | — | | — | | — | | 1 | (16.7) | — | | 1 | (5.0) |
| adenocarcinoma | | | | | | | | | | | | |
| Non-small cell lung cancer | 2 | (40.0) | — | | 1 | (33.3) | — | | 1 | (33.3) | 4 | (20.0) |
| Renal cell carcinoma | — | | — | | — | | — | | 1 | (33.3) | 1 | (5.0) |
| Squamous cell carcinoma - | — | | — | | — | | 1 | (16.7) | — | | 1 | (5.0) |
| head and neck | | | | | | | | | | | | |
| Prior Treatments for Cancer, n | | | | | | | | | | | | |
| (%) | | | | | | | | | | | | |
| Yes | 5 | (100) | 3 | (100) | 3 | (100) | 6 | (100) | 3 | (100) | 20 | (100) |
| Number of Prior Treatments for | | | | | | | | | | | | |
| Cancer, n (%) | | | | | | | | | | | | |
| Mean (StD) | 4.0 | (2.1) | 3.3 | (1.2) | 4.0 | (2.6) | 3.8 | (1.7) | 4.0 | (1.7) | 3.9 | (1.7) |
| Median | 4 | | 4 | | 3 | | 4 | | 3 | | 3.5 | |
| Minimum, Maximum | 2, 7 | | 2, 4 | | 2, 7 | | 2, 6 | | 3, 6 | | 2, 7 | |
| Time from Diagnosis (months) | | | | | | | | | | | | |
| Mean (StD) | 31.9 | (18.9) | 49.0 | (8.8) | 22.9 | (22.6) | 40.6 | (15.1) | 70.4 | (43.3) | 41.5 | (24.8) |

| | | | | | | |
|---|---|---|---|---|---|---|
| Median | 28.7 | 44.2 | 12.7 | 42.4 | 53.2 | 42.4 |
| Minimum, Maximum | 10.9, 62.5 | 43.7, 59.2 | 7.2, 48.8 | 22.7, 65.1 | 38.5, 119.7 | 7.2, 119.7 |

### A. Safety Results

Preliminary data indicate that the study treatments were generally well-tolerated. Of the 130 reported AEs, 21 were considered possibly related to the study treatment. Most events were mild to moderate and unrelated to study treatment. Only one Grade 3 event of hepatic enzyme increased (1 mg/kg), measured at the start of Cycle 6, was considered possibly related to the administered drug. Due to hospitalization, the event was considered a serious adverse event (SAE). The patient recovered within 3 days without requiring drug or non-drug therapy. No DLT or grade 4 or 5 events were documented. Apart from the above-mentioned SAE, no other drug-related SAEs were reported. No AEs were the direct cause of patient death.

AEs considered possibly related to SAR444881 treatment included anemia, abdominal distension, constipation, diarrhea, flatulence, nausea, amylase increased, blood creatine phosphokinase increased, hepatic enzyme increased, hyponatremia and oropharyngeal pain, each reported for 1 patient and abdominal pain, fatigue and pruritis, each reported for 2 patients. No events were considered possibly related to the highest tested dose level (10 mg/kg SAR444881). No events were considered possibly related to the highest tested dose level (10 mg/kg SAR444881).

Clinically significant hematological abnormalities were measured in 3 patients, clinically significant blood biochemistry abnormalities were measured in 6 patients and clinically significant coagulation abnormalities were identified in one patient. All were considered non-treatment-related, except for amylase increased (0.1 mg/kg patient), blood creatine phosphokinase increased (0.3 mg/kg patient) and hepatic enzyme increased (1 mg/kg patient).

### B. Pharmacokinetic and Pharmacodynamic Results

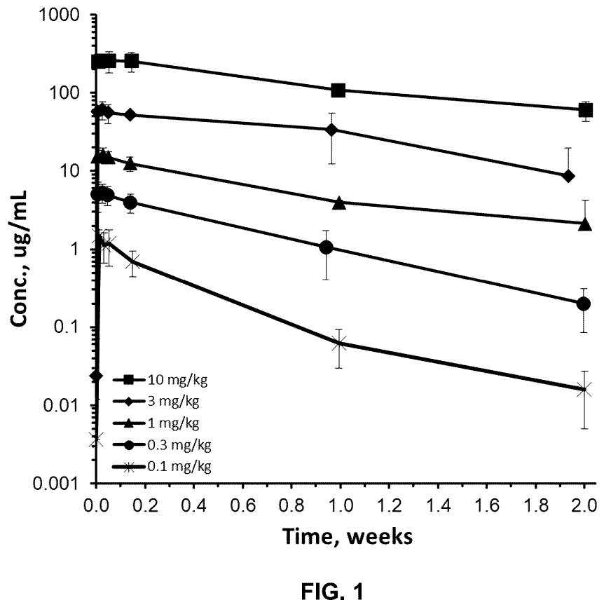

SAR444881 PK proved to be consistent within cohorts but dose-dependent, with Cₘₐₓ and AUC increasing and elimination rate declining with increasing doses. No SAR444881 accumulation was apparent between doses. These dose-dependent differences in the pharmacokinetics of SAR444881 strongly suggest target-mediated drug disposition (TMDD) of SAR444881, i.e., increasing extent of saturation of the endogenous pool of ILT2 protein with increases in the drug dose. See FIG. 1 and Table 4 below:

TABLE 4
SAR444881 Pharmacokinetics

| | Treatment Group | | | | |
|---|---|---|---|---|---|
| | 0.1 | 0.3 | 1 | 3 | 10 |
| | mg/kg | mg/kg | mg/kg | mg/kg | mg/kg |
| | (N = 5) | (N = 3) | (N = 3) | (N = 4) | (N = 3) |
| Tₘₐₓ, (wk) | | | | | |
| Mean (SD) | 0.02 | 0.02 | 0.01 | 0.03 | 0.10 |
| | (0.02) | (0.01) | (0.01) | (0.02) | (0.08) |
| % CV | 75 | 60 | 67 | 63 | 80 |
| Cₘₐₓ, (μg/ml) | | | | | |
| Mean (SD) | 1.62 | 5.57 | 16.93 | 64.80 | 276.67 |
| | (0.33) | (1.71) | (2.49) | (11.56) | (48.18) |
| % CV | 20 | 31 | 15 | 18 | 17 |
| Terminal | | | | | |
| T_{1/2}, (wk) | | | | | |
| Mean (SD) | 0.306 | 0.417 | 0.717 | 1.162 | 1.144 |
| | (0.077) | (0.049) | (0.383) | (1.003) | (0.510) |
| % CV | 25 | 12 | 53 | 86 | 45 |
| AUC₀₋ₜ, | | | | | |
| (wk * g/mL) | | | | | |
| Mean (SD) | 0.527 | 3.33 | 13.81 | 98.21 | 303.4 |
| | (0.188) | (1.169) | (4.65) | (101.9) | (13.3) |
| % CV | 36 | 35 | 34 | 104 | 4 |
| Vₛₛ, (mL/kg) | | | | | |
| Mean (SD) | 59.2 | 43.0 | 53.6 | 43.1 | 38.4 |
| | (40.4) | (7.8) | (13.1) | (14.6) | (14.2) |
| % CV | 68 | 18 | 24 | 34 | 37 |
| CL | | | | | |
| (mL/kg/wk) | | | | | |
| Mean (SD) | 210 | 96 | 69 | 42 | 25 |
| | (85) | (37) | (29) | (26) | (3) |
| % CV | 40 | 39 | 42 | 61 | 13 |

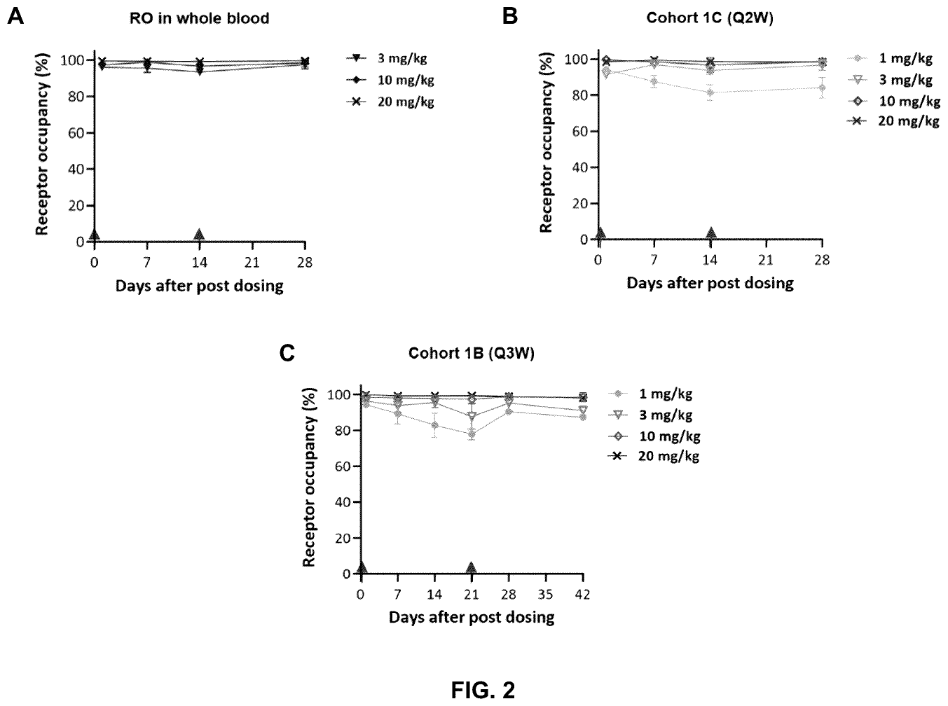

ILT2 receptor occupancy (RO) by SAR444881 was examined in fresh whole blood samples of patients enrolled to 3, 10 and 20 mg/kg dose level cohorts. RO was measured using the “free” indirect method for assessing the proportion of unoccupied (unbound) sites utilizing a phycoerythrin (PE)-labeled antibody that competes with the drug molecule. The analysis was performed on peripheral monocytes collected from patients, which represent the immune population which has the highest ILT2 expression among all peripheral immune cells. The results depicted in FIG. 2, Panel A demonstrate high initial levels of RO after the first administration of SAR444881 (C1D2-3 mg/kg, 96.13+0.96; 10 mg/kg 97.4+1.81; 20mg/kg 99.5+1.07).

Further, measurement of RO levels in patients that received monotherapy of SAR444881 at dose levels above 3 mg/kg demonstrated target saturation and sustained occupancies for SAR444881 at dose level of 3 mg/kg and up at a Q2W based regimen. In support of this, results observed for patients receiving administration of 1, 3, 10 and 20 mg/kg of SAR444881 in combination with cetuximab (Q2W regimen) or pembrolizumab (Q3W regimen) demonstrated sustained saturation at SAR444881 doses that were equal to or higher than 3 mg/kg (FIG. 2, Panels B and C).

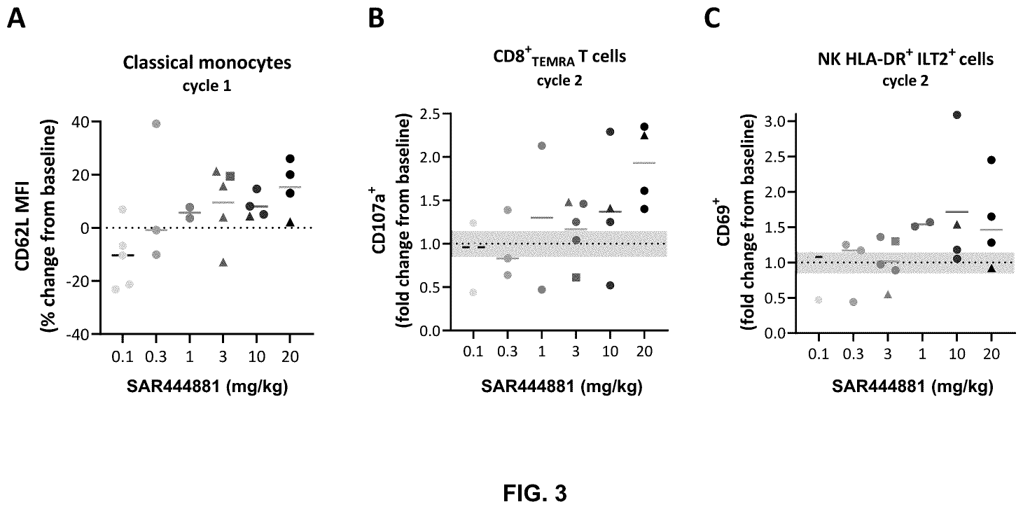

Evidence for the immunomodulatory activity of SAR444881 was observed in both myeloid and lymphocyte compartments. Patients treated with saturating doses of SAR444881 (≥3 mg/kg) showed elevated levels of the CD62L marker on the surface of classic monocytes (FIG. 3, Panel A). CD62L, also known as L-selectin, is a cell adhesion molecule that plays a role in regulating the recruitment of monocytes to tissues from the blood during inflammation. In parallel, upregulation of immune activation markers was seen in the lymphocyte population, such as CD107a and CD69 on ILT2-expressing T and NK cell subsets, following SAR444881 treatment (FIG. 3, Panels B and C).

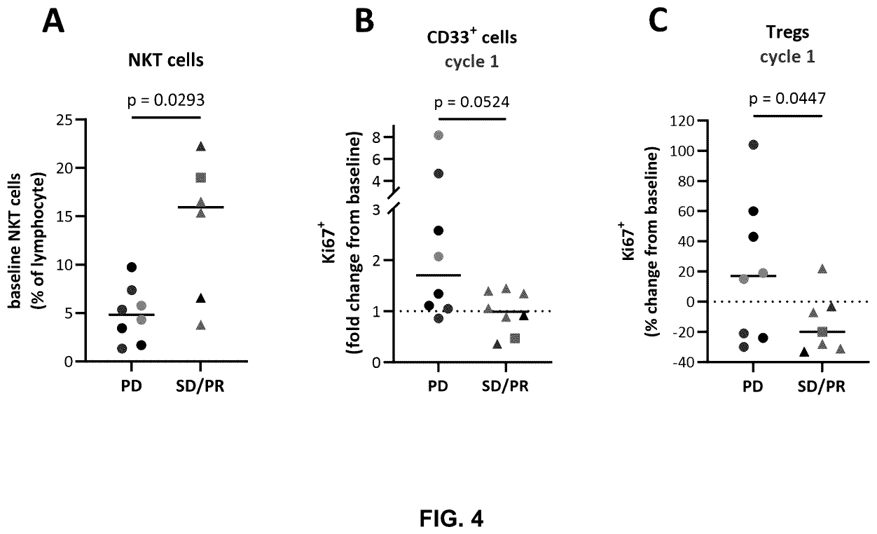

Analysis also demonstrated that patients with a clinical benefit (PR or SD, vs. PD) had statistically significantly higher baseline levels of ILT2-positive NKT cells (FIG. 4, Panel A). In addition, an association was observed between clinical response to SAR444881treatment and reduced proliferation of myeloid cell subsets (p=0.052, one-tailed Mann-Whitney test) and regulatory T cells (p=0.0447, one tailed T-test) (FIG. 4, Panels B and C).

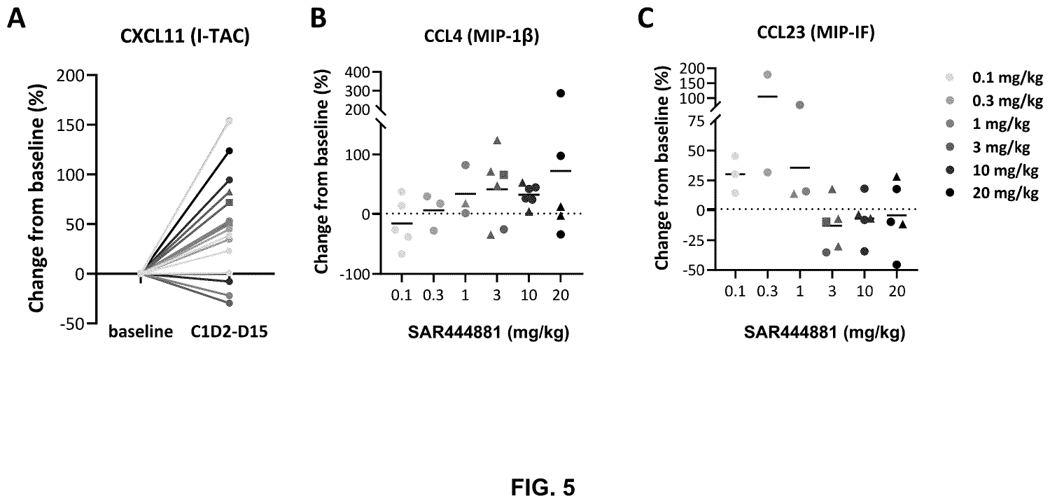

Serum samples from patients enrolled in the study were analyzed for the presence of 16 different chemokines and cytokines. An elevation compared to baseline levels was observed for CXCL11 and CCL4 during the first cycle of SAR444881 treatment (FIG. 5, Panels A and B), while a reduction trend was observed for CCL23 during cycle 2 of SAR444881 treatment (FIG. 5, Panel C). Patients treated with SAR444881 dose levels≥1 mg/kg (14 out of 20 patients, 70%) demonstrated a dose-response trend in changes from baseline in serum levels of CCL4 and CCL23 (FIG. 5, Panels B and C). A decrease in CCL23 was observed in 11 of 15 subjects (73.3%) treated with SAR444881≥3 mg/kg (FIG. 5, Panel C). These chemokines are known to be produced by myeloid cells and to exert various activities in the immune system, such as cell recruitments and promotion of inflammatory conditions. Of note, evaluation of the pre-treatment biopsy of patient who achieved PR revealed strong CD8 expression with high stromal and tumor-infiltrating CD8 -positive cells, combined with high levels of intra-tumoral ILT2 expression (90%).

### C. Efficacy Results

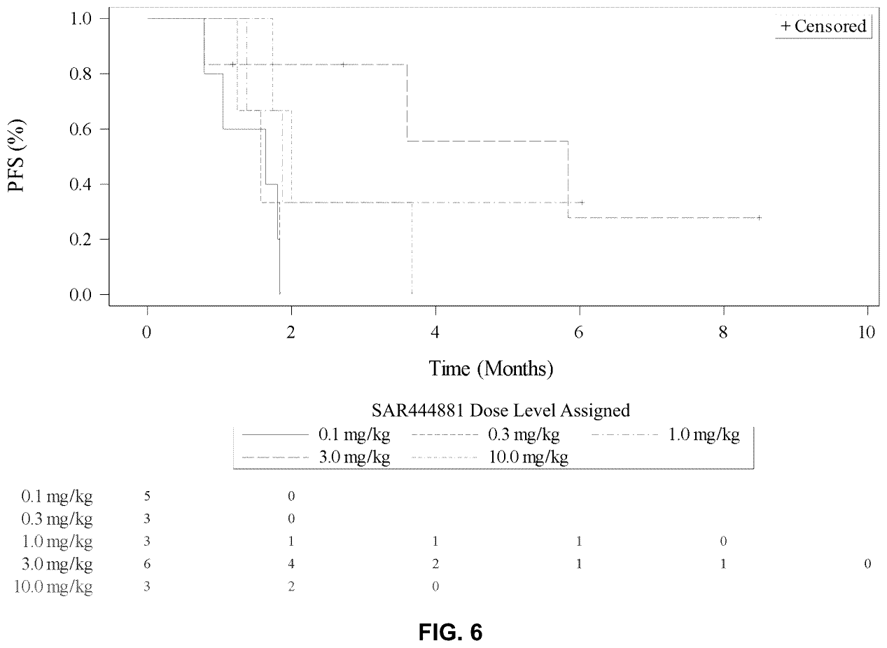

One patient, treated with 3 mg/kg SAR444881, showed a partial response. The patient was still on treatment (Cycle 9) at the interim cutoff date, with a duration of response of 13.3 weeks. Stable disease (SD) was documented in 1 (33.3%) patient receiving 1 mg/kg SAR444881, 3 (50%) patients receiving 3 mg/kg SAR444881 and 1 (33.3%) patient receiving 10 mg/kg SAR444881. A best overall response of progressive disease (PD) was documented for 11 (55%) patients. Taken together, the ORR for the 3 mg/kg dose level was 16.7% and for the entire Sub-Part 1A population was 5%. The disease control rate, defined as best overall response of CR, PR, or SD, for the 1 mg/kg, 3 mg/kg, and 10 mg/kg cohorts, was 33.3%, 66.7% and 33.3%, respectively and was 30% for the entire Sub-Part 1A population. Median progression-free survival (PFS) time, defined as the time from the date of first dose of study drug to the date of first documented disease progression or death due to any cause, was 1.8 months across all dose levels and ranged between 1.6 months (0.1 and 0.3 mg/kg cohorts) and 5.8 months (3 mg/kg cohort) (FIG. 6). Overall PFS rate at 3 months was 31.9% and ranged between 20.0% (0.1 mg/kg cohort) and 83.3% (3 mg/kg cohort).

### D. Conclusions

The results of this analysis of first-in-human testing of SAR444881 monotherapy at doses of up to 10 mg/kg in 20 patients with advanced solid tumors, who were either refractory to or unsuitable for available treatments, demonstrate its favorable safety profile, including at the highest dose level tested so far.

Partial response (n=1) and stable disease assessments (n=5) were reported at SAR444881 doses of 1 mg/kg and above. The disease control rate was 30%. The one patient who had achieved a partial response (3 mg/kg cohort) was on treatment for 39 weeks at the preliminary data cutoff date, with a duration of response to date of 13.3 weeks. Median progression-free survival for the entire population was 1.8 months.

PK analyses suggested an increasing extent of saturation with increasing doses, manifesting by higher maximum serum concentrations, longer half-life, slower clearance and larger volume of distribution. PK exposure of SAR444881 was nearly linear and dose proportional from 2 mg/kg to 20 mg/kg in both Q2W and Q3W regimens based on population pharmacokinetic (PopPK) modeling. The PopPK/PD modeling suggested that maintaining a median target receptor occupancy (RO) exceeding 90% was achievable at 3 mg/kg or higher in both Q2W and Q3W regimens. Sustained saturated peripheral monocyte ILT2 receptor occupancy throughout the course of treatment, was noted in patients administered 3 mg/kg and 10 mg/kg of SAR444881. Preliminary analyses found that patients with a clinical benefit (PR or SD) had statistically significantly higher baseline levels of ILT2-positive NKT cells (p=0.0293). and treatment-associated downregulation of CCL7. Of note, high baseline levels of intra-tumoral ILT2 expression (90%) were seen in the patient who achieved a PR, along with significant increases in the levels of the cytokines CCL4, CXCL11, CCL23 and granzyme B. Analyses also demonstrated an upregulation of immune activation markers, such as CD69 and CD107a, on ILT2-expressing NK and T cells subsets and of the adhesion molecule CD62L on monocytes following SAR444881 treatment.

In summary, SAR444881 monotherapy appeared to be safe and well tolerated at doses up to 10 mg/kg. Preliminary signs of anti-tumor activity in this heavily pretreated, advanced cancer patient population were observed.

### Example 4: Safety and Efficacy Data for SAR444881 Monotherapy and Combination Treatment (Sub-Parts 1A, 1B, and 1C)

This Example describes preliminary safety, pharmacokinetic, and efficacy data for Part 1-Sub-Parts 1A-1C of the clinical study as described in Example 1.

The primary objectives of the study were to assess the safety and tolerability of SAR444881 when administered alone or in combination with pembrolizumab or cetuximab. The secondary objectives of the study were to assess the preliminary anti-tumor activity and to characterize the PK and immunogenicity of the treatments.

This study enrolled 76 patients (31 in Sub-Part 1A, 23 in Sub-Part 1B, and 22 in Sub-Part 1C) who had received ≥1 doses of SAR444881.

### A. Results

The average duration of treatment across Sub-Parts 1A, 1B, and 1C was 11 weeks. The most common treatment-related adverse events (AEs) were fatigue (19%) for Sub-Part 1A, nausea (17%) for Sub-Part 1B, and rash (50%) for Sub-Part 1C. Adverse events (AEs) in Sub-Part 1C included: rash in 11 patients (50%), dry skin in 7 patients (32%), fatigue in 7patients (32%), and dermatitis acneiform in 6 patients (27%). Serious treatment emergent AEs (TEAEs) were reported in 20 patients (26%), four of which were deemed to be treatment-related (see Table 5 below). No treatment-related dose-limiting toxicities or life-threatening/fatal treatment-related AEs were reported.

In assessing anti-tumor activity, one patient in Sub-Part 1A (3%), one patient in Sub-Part 1B (4%), and two patients in Sub-Part 1C (9%) achieved a partial response (see Table 5 below). The median progression free survival (PFS) in months was 1.8 (1.6-3.4) for Sub-Part 1A; 1.6 (1.5-2.1) for Sub-Part 1B; and 3.9 (1.8-NC) for Sub-Part 1C.

The 3 mg/kg and 10 mg/kg doses were identified as safe and exhibited anti-tumor activity, achieving high target receptor occupancy (>93%). All responders at 3-10 mg/kg bore tumors predicted to be insensitive or progressing after anti-PD-1 and/or cetuximab treatment. SAR444881 treatment at doses of ≥3 mg/kg led to upregulation of activation markers in monocytes and ILT2-expressing T and NK cell subsets. An increase in intratumoral necrosis and infiltration of CD8 T cell subsets was observed.

Based on these data, 10 mg/kg may be used as the initial dose of SAR444881 for dose optimization.

TABLE 5
SAR444881 Safety and Efficacy

| | | | Sub-Part 1B | | Sub-Part 1C | |
|---|---|---|---|---|---|---|
| | Sub-Part 1A | | SAR444881 + | | SAR444881 + | |
| | SAR444881 | | Pembrolizumab | | Cetuximab | |
| | (n = 31) | | (n = 23) | | (n = 22) | |
| Safety; n (%) | | | | | | |
| Any grade AEs | 30 | (97) | 23 | (100) | 22 | (100) |
| Grade 3-5 TEAEs | 14 | (45) | 9 | (39) | 6 | (27) |
| Serious TEAEs | 7 | (23)^{a} | 8 | (35)^{b} | 5 | (23)^{c} |
| Best overall | | | | | | |
| response*; n (%) | | | | | | |
| Partial response | 1 | (3)^{d} | 1 | (4)^{e} | 2 | (9)^{e} |
| Stable disease | 9 | (29) | 4 | (17) | 9 | (41) |
| Progressive disease | 17 | (55) | 14 | (61) | 8 | (36) |
| Not evaluable | 4 | (13) | 4 | (17) | 3 | (14) |

Treatment-related SAEs:
aOne SAE (elevated liver enzymes)
^{b}One SAE (back pain)
^{c}Two Grade 2 infusion-related reactions.
Tumor types:
^{d}Gastroesophageal junction carcinoma
^{e}Colorectal cancer.
*Per RECIST v1.1.
AE, adverse event; SAE, serious AE, TEAE, treatment emergent AE.

### B. Conclusions

Treatment with SAR444881. alone or in combination with pembrolizumab or cetuximab, was well tolerated in patients with advanced solid tumors.

1-42. (canceled)

43. A method of treating cancer in a human patient in need thereof, comprising administering to the patient a humanized monoclonal anti-human ILT2 IgG₄ antibody whose heavy chain CDR1-3 and light chain CDR1-3 comprise SEQ ID NOs: 1-6, respectively, in two or more treatment cycles, wherein each treatment cycle comprises a single dose of the anti-ILT2 antibody at 0.1-20 mg/kg per dose.

44. The method of claim 43, wherein
a) the dose is 0.1, 0.3, 1, 3, 10, or 20 mg/kg;
b) each treatment cycle is about two or three weeks; or
c) a) and b).

45. The method of claim 43, wherein the antibody comprises
a) a heavy chain variable domain and a light chain variable domain comprising SEQ ID NOs: 7 and 8, respectively; or
b) a heavy chain comprising SEQ ID NO: 9, or SED ID NO: 9 without the C-terminal lysine; and a light chain comprising SEQ ID NO: 10.

46. The method of claim 43, wherein the cancer is selected from the group consisting of breast cancer, triple negative breast cancer, biliary tract cancer, cervical cancer, cholangiocarcinoma, colorectal cancer, esophageal cancer, gastric cancer, gastroesophageal junction adenocarcinoma, hepatobiliary cancer, hepatocellular carcinoma, head and neck cancer, non-small cell lung cancer, renal cell carcinoma, skin squamous cell carcinoma, ovarian cancer, pancreatic cancer, renal cell carcinoma, and urothelial cancer.

47. The method of claim 43, wherein the antibody is administered at a first dose and a second dose, wherein
a) the first dose is higher than the second dose;
b) the second dose is higher than the first dose;
c) the first dose is 20 mg/kg, and the second dose is 1 mg/kg;
d) the first dose is 10 mg/kg, and the second dose is 1 mg/kg;
e) the first dose is 3 mg/kg, and the second dose is 1 mg/kg;
f) the first dose is 1 mg/kg, and the second dose is 0.3 mg/kg; or
g) the first dose is 0.3 mg/kg, and the second dose is 0.1 mg/kg.

48. The method of claim 43, wherein the antibody is administered to the patient by intravenous infusion.

49. The method of claim 43, further comprising administering to the patient in each treatment cycle
a) a single dose of pembrolizumab;
b) a) with chemotherapy;
c) a) with carboplatin;
d) a) with pemetrexed; or
e) a) with carboplatin and pemetrexed;
f) d) or e) with premedication with folic acid, vitamin B12, dexamethasone, or any combination thereof before each administration of pemetrexed.

50. The method of claim 49, wherein
a) the pembrolizumab is administered intravenously at 200 mg per dose;
b) the carboplatin is administered intravenously to an AUC of 5 per treatment cycle;
c) the pemetrexed is administered intravenously at a dose of 500 mg/m² per treatment cycle; or
d) any combination thereof.

51. The method of claim 49, wherein each treatment cycle is about three weeks.

52. The method of claim 43, further comprising administering to the patient in each treatment cycle
a) a single dose of cetuximab; or
b) a) with premedication with a histamine-1 (H1) receptor antagonist before each administration of cetuximab.

53. The method of claim 52, wherein the cetuximab is administered intravenously at 500 mg/m² per dose.

54. The method of claim 52, wherein the Hi receptor antagonist is diphenhydramine.

55. The method of claim 52, wherein each treatment cycle is about two weeks.

56. The method of claim 43, wherein said treatment is
a) first line therapy;
b) second line therapy; or
c) third line or later therapy.

57. The method of claim 43,
wherein the cancer is breast cancer, biliary tract cancer, cervical cancer, colorectal cancer, esophageal cancer, gastric cancer, gastroesophageal junction adenocarcinoma, hepatocellular carcinoma, head and neck cancer, non-small cell lung cancer, renal cell carcinoma, skin squamous cell carcinoma, urothelial cancer, or cholangiocarcinoma,
wherein the anti-ILT2 antibody comprises a heavy chain and a light chain having the amino acid sequences of SEQ ID NOs: 9 and 10, respectively, and
wherein the antibody is administered by intravenous infusion in two or more treatment cycles, wherein each treatment cycle comprises a single dose of the anti-ILT2 antibody at 0.1, 0.3, 1, 3, 10 or 20 mg/kg per dose, wherein each treatment cycle is about two weeks, optionally wherein the treatment is second line or later therapy.

58. The method of claim 43,
wherein the cancer is biliary tract cancer, cervical cancer, colorectal cancer, esophageal cancer, gastric cancer, gastroesophageal junction adenocarcinoma, hepatocellular carcinoma, head and neck cancer, non-small cell lung cancer, pancreatic cancer, renal cell carcinoma, skin squamous cell carcinoma, triple negative breast cancer, or urothelial cancer,
wherein the anti-ILT2 antibody comprises a heavy chain and a light chain having the amino acid sequences of SEQ ID NOs: 9 and 10, respectively, and
wherein the method further comprises the step of administering pembrolizumab to the patient,
wherein the antibody and pembrolizumab are administered by intravenous infusion in two or more treatment cycles, wherein each treatment cycle comprises a single dose of the anti-ILT2 antibody at 0.1, 0.3, 1, 3, 10 or 20 mg/kg per dose, and a single 200 mg dose of pembrolizumab, wherein each treatment cycle is about three weeks,
optionally wherein the treatment is second or third line therapy.

59. The method of claim 43, wherein the cancer is non-squamous non-small cell lung cancer,
wherein the anti-ILT2 antibody comprises a heavy chain and a light chain having the amino acid sequences of SEQ ID NOs: 9 and 10, respectively,
wherein the method further comprises the step(s) of administering pembrolizumab, carboplatin, and pemetrexed to the patient, wherein the antibody, pembrolizumab, carboplatin, and pemetrexed are administered by intravenous infusion in two or more treatment cycles, wherein each treatment cycle comprises
i) a single dose of the anti-ILT2 antibody at 1, 3, 10 or 20 mg/kg per dose,
a single 200 mg dose of pembrolizumab,
a single dose of carboplatin to AUC of 5, and
a single 500 mg/m² dose of pemetrexed, or
ii) i) with pretreatment with folic acid, vitamin B12, dexamethasone, or any combination thereof before each administration of pemetrexed,
wherein each treatment cycle is about three weeks, optionally wherein the treatment is first line therapy.

60. The method of claim 43,
wherein the cancer is colorectal cancer, head and neck cancer, non-small cell lung cancer, ovarian cancer, or hepatobiliary cancer,
wherein the anti-ILT2 antibody comprises a heavy chain and a light chain having the amino acid sequences of SEQ ID NOs: 9 and 10, respectively, and
wherein the method further comprises the step of administering to the patient
a) cetuximab, or
b) a) with premedication with diphenhydramine before each administration of cetuximab,
wherein the antibody and cetuximab are administered by intravenous infusion in two or more treatment cycles, wherein each treatment cycle comprises a single dose of the anti-ILT2 antibody at 0.1, 0.3, 1, 3, 10 or 20 mg/kg per dose, and a single 500 mg/m² dose of cetuximab, wherein each treatment cycle is about two weeks.

61. The method of claim 43,
wherein the cancer is colorectal cancer or non-small lung cancer,
wherein the anti-ILT2 antibody comprises a heavy chain and a light chain having the amino acid sequences of SEQ ID NOs: 9 and 10, respectively, and wherein the method further comprises administering cetuximab to the patient, wherein the antibody and cetuximab are administered by intravenous infusion in two or more treatment cycles, wherein each treatment cycle comprises
a) a single dose of the anti-ILT2 antibody at 1, 3, 10 or 20 mg/kg per dose, and a single 500 mg/m² dose of cetuximab, or
b) a) with pretreatment with diphenhydramine at a dose of 50 mg before each administration of cetuximab,
wherein each treatment cycle is about two weeks, and optionally wherein the treatment is second line or later therapy.

62. The method of claim 43, wherein
a) the cancer is unresectable;
b) the cancer is metastatic;
c) the patient is refractory to standard approved therapy;
d) the patient is not a candidate for standard approved therapy; or
e) any combination of a)-d).


SEQUENCE LISTING (23 sequences)

SEQ ID NO: 1 - protein, 6 aa, synthetic construct
  REGION 1..6: note = Synthetic
SGYYWN

SEQ ID NO: 2 - protein, 16 aa, synthetic construct
  REGION 1..16: note = Synthetic
YISYDGSNNYNPSLKN

SEQ ID NO: 3 - protein, 10 aa, synthetic construct
  REGION 1..10: note = Synthetic
GYSYYYAMDA

SEQ ID NO: 4 - protein, 11 aa, synthetic construct
  REGION 1..11: note = Synthetic
RTSQDISNYLN

SEQ ID NO: 5 - protein, 7 aa, synthetic construct
  REGION 1..7: note = Synthetic
YTSRLHS

SEQ ID NO: 6 - protein, 8 aa, synthetic construct
  REGION 1..8: note = Synthetic
QQGNTLPT

SEQ ID NO: 7 - protein, 119 aa, synthetic construct
  REGION 1..119: note = Synthetic
DVQLQGSGPGLVKPSETLSLTCSVTGYSITSGYYWNWIRQFPGKKLEWMGYISYDGSNNYNPSLKNRITISRDTSKNQFSLKLNSVTAADTATYYCAHGYSYYYAMDAWGQGTSVTVSS

SEQ ID NO: 8 - protein, 106 aa, synthetic construct
  REGION 1..106: note = Synthetic
DIQMTQSPSSLSASVGDRVTITCRTSQDISNYLNWYQQKPGKAVKLLISYTSRLHSGVPSRFSGSGSGTDYTLTISSLQPEDFATYYCQQGNTLPTFGQGTKLEIK

SEQ ID NO: 9 - protein, 446 aa, synthetic construct
  REGION 1..446: note = Synthetic
DVQLQGSGPGLVKPSETLSLTCSVTGYSITSGYYWNWIRQFPGKKLEWMGYISYDGSNNYNPSLKNRITISRDTSKNQFSLKLNSVTAADTATYYCAHGYSYYYAMDAWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 213 aa, synthetic construct
  REGION 1..213: note = Synthetic
DIQMTQSPSSLSASVGDRVTITCRTSQDISNYLNWYQQKPGKAVKLLISYTSRLHSGVPSRFSGSGSGTDYTLTISSLQPEDFATYYCQQGNTLPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, synthetic construct
  REGION 1..8: note = Synthetic
GYSITSGY

SEQ ID NO: 12 - protein, 5 aa, synthetic construct
  REGION 1..5: note = Synthetic
SYDGS

SEQ ID NO: 13 - protein, 9 aa, synthetic construct
  REGION 1..9: note = Synthetic
GYSITSGYY

SEQ ID NO: 14 - protein, 7 aa, synthetic construct
  REGION 1..7: note = Synthetic
ISYDGSN

SEQ ID NO: 15 - protein, 12 aa, synthetic construct
  REGION 1..12: note = Synthetic
AHGYSYYYAMDA

SEQ ID NO: 16 - protein, 7 aa, synthetic construct
  REGION 1..7: note = Synthetic
TSGYYWN

SEQ ID NO: 17 - protein, 12 aa, synthetic construct
  REGION 1..12: note = Synthetic
WMGYISYDGSNN

SEQ ID NO: 18 - protein, 11 aa, synthetic construct
  REGION 1..11: note = Synthetic
AHGYSYYYAMD

SEQ ID NO: 19 - protein, 6 aa, synthetic construct
  REGION 1..6: note = Synthetic
QDISNY

SEQ ID NO: 20 - skipped

SEQ ID NO: 21 - protein, 7 aa, synthetic construct
  REGION 1..7: note = Synthetic
SNYLNWY

SEQ ID NO: 22 - protein, 10 aa, synthetic construct
  REGION 1..10: note = Synthetic
LLISYTSRLH

SEQ ID NO: 23 - protein, 7 aa, synthetic construct
  REGION 1..7: note = Synthetic
QQGNTLP